SEQUENCE LISTING (28 sequences)

SEQ ID NO: 1 - protein, 192 aa, Homo sapiens
AAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPN

SEQ ID NO: 2 - protein, 28 aa, Homo sapiens
YRVLLGQLQKQTDLMQDTSRLLDPYIRI

SEQ ID NO: 3 - protein, 25 aa, Homo sapiens
GRRGFLQTLNATLGCVLHRLADLEQ

SEQ ID NO: 4 - protein, 26 aa, Homo sapiens
EDLEKLQMARPNILGLRNNIYCMAQL

SEQ ID NO: 5 - protein, 27 aa, Homo sapiens
AFQRKLEGCRFLHGYHRFMHSVGRVFS

SEQ ID NO: 6 - protein, 187 aa, Homo sapiens
AAIGSCSKEYRVLLGQLQKQTDLMQDTSRLLDPYIRIQGLDVPKLREHCRERPGAFPSEETLRGLGRRGFLQTLNATLGCVLHRLADLEQRLGAPSGLNIEDLEKLQMARPNILGLRNNIYCMAQLLDNSDTAEPTKAGAGASQPPTPTPASDAFQRKLEGCRFLHGYHRFMHSVGRVFSKWGESPN

SEQ ID NO: 7 - protein, 184 aa, Homo sapiens
AAIGSCSKEYRVLLGQLQKQTDLMQDTSRLLDPYIRIQGLDVPKLREHCRERPGAFPSEETLRGLGRRGFLQTLNATLGCVLHRLADLEQRLGGGNIEDLEKLQMARPNILGLRNNIYCMAQLLDNSDTAEPTKAGAGASQPPTPTPASDAFQRKLEGCRFLHGYHRFMHSVGRVFSKWGESPN

SEQ ID NO: 8 - protein, 180 aa, Homo sapiens
AAIGSCSKEYRVLLGQLQKQTDLMQDTSRLLDPYIRIQGLDVPKLREHCRERPGAFPSEETLRGLGRRGFLQTLNATLGCVLHRLADLEQRNIEDLEKLQMARPNILGLRNNIYCMAQLLDNSDTAEPTKAGAGASQPPTPTPASDAFQRKLEGCRFLHGYHRFMHSVGRVFSKWGESPN

SEQ ID NO: 9 - protein, 12 aa, Homo sapiens
LPKAQDLERSGL

SEQ ID NO: 10 - protein, 7 aa, Homo sapiens
LGAPSGL

SEQ ID NO: 11 - protein, 4 aa, Homo sapiens
LGGG

SEQ ID NO: 12 - protein, 4 aa, Homo sapiens
  misc_feature (2)..(3): Xaa can be any naturally occurring amino acid
FXXK

SEQ ID NO: 13 - protein, 4 aa, Homo sapiens
  misc_feature (2)..(3): Xaa can be any naturally occurring amino acid
AXXA

SEQ ID NO: 14 - DNA, 33 bp, Homo sapiens
ctggttccgcgtggatccgcggctataggcagc

SEQ ID NO: 15 - DNA, 33 bp, Homo sapiens
cagtcacgatgaattcgactatctccggctccg

SEQ ID NO: 16 - DNA, 28 bp, Homo sapiens
cacgaaggctggcgcgggggcctctcag

SEQ ID NO: 17 - DNA, 28 bp, Homo sapiens
ctgagaggcccccgcggcagccttcgtg

SEQ ID NO: 18 - DNA, 39 bp, Homo sapiens
ccctgcctcggatgctgctcagcgcgcgctggagggctg

SEQ ID NO: 19 - DNA, 39 bp, Homo sapiens
cagccctccagcgcgcgctgagcagcatccgaggcaggg

SEQ ID NO: 20 - DNA, 43 bp, Homo sapiens
gacttagagcagcgcctcggcgcgccccaggatttggagaggt

SEQ ID NO: 21 - DNA, 43 bp, Homo sapiens
acctctccaaatcctggggcgcgccgaggcgctgctctaagtc

SEQ ID NO: 22 - DNA, 24 bp, Homo sapiens
ctcggcgcgccctctgggctgaac

SEQ ID NO: 23 - DNA, 24 bp, Homo sapiens
gttcagcccagagggcgcgccgag

SEQ ID NO: 24 - DNA, 30 bp, Homo sapiens
cagcgcctcggcgggggctctgggctgaac

SEQ ID NO: 25 - DNA, 30 bp, Homo sapiens
gttcagcccagagcccccgccgaggcgctg

SEQ ID NO: 26 - DNA, 27 bp, Homo sapiens
cctcggcgggggcaacatcgaggactt

SEQ ID NO: 27 - DNA, 27 bp, Homo sapiens
aagtcctcgatgttgcccccgccgagg

SEQ ID NO: 28 - protein, 192 aa, Homo sapiens; identical to SEQ ID NO: 1

(19) United States
(12) Patent Application Publication
(10) Pub. No.: US 2010/0310509 A1
(43) Pub. Date: Dec. 9, 2010

(54) COMPOSITIONS COMPRISING MODULATORS OF SIGNAL TRANSDUCING RECEPTOR gp130 AND METHODS OF PRODUCING AND USING SAME

(76) Inventors: JOHN D. ASH, EDMOND, OK (US); SRINIVAS CHOLLANGI, PLANO, TX (US)

Correspondence Address: DUNLAP CODDING, P.C., PO BOX 16370, OKLAHOMA CITY, OK, 73113, US

(21) Appl. No.: 12/794,368

(22) Filed: Jun. 4, 2010

Related U.S. Application Data
(60) Provisional application No. 61/217,780, filed on Jun. 4, 2009.

(51) Int. Cl. A61K 38/20; C07H 21/04; C07K 14/54; A61P 3/04; A61P 3/10; A61P 1/18; A61P 9/12; A61P 29/00; A61P 19/02; A61P 19/06; A61P 35/00; A61P 3/06; C12N 15/74; C12N 5/10

(52) U.S. Cl. 424/85.2; 536/23.5; 530/351; 435/320.1; 435/325; 435/375

(57) ABSTRACT

Compositions comprising modulators of gp130, as well as methods of producing and using same, are disclosed.

### CROSS REFERENCE TO RELATED APPLICATIONS

This application claims benefit under 35 U.S.C. 119(e) of U.S. Ser. No. 61/217,780, filed Jun. 4, 2009. The entire contents of the above-referenced application are hereby expressly incorporated herein by reference.

### STATEMENT REGARDING FEDERALLY SPONSORED RESEARCH OR DEVELOPMENT

This invention was made with government support under Grant Nos. P20RR017703-07 and EY016459 awarded by the National Institutes of Health. The government has certain rights in the invention.

### BACKGROUND OF THE PRESENTLY DISCLOSED AND CLAIMED INVENTIVE CONCEPT(S)

1. Field of the Presently Disclosed and Claimed Inventive Concept(s)

The presently disclosed and claimed inventive concept(s) relates generally to compositions comprising modulators of signal transducing receptors, and in particular, but not by way of limitation, to compositions comprising modulators of gp130, and methods of producing and using same.

2. Description of the Background Art

The IL-6 family of cytokines comprise interleukin-6 (IL-6), interleukin-11 (IL-11), leukemia inhibitory factor (LIF), Oncostatin M (OSM), ciliary neurotrophic factor (CNTF), cardiotrophin-1 (CT-1) and cardiotrophin-like cytokine (CLC). These cytokines possess a typical “four-α-helix bundle”-like structure and act on their target cells by forming a multimeric receptor complex which includes a common receptor glycoprotein 130 (gp130). Extensive mutagenesis studies revealed that IL-6 type cytokines interact with the receptor-chains through three distinct binding sites referred to as sites I, II, and III. Cytokines requiring a co-receptor chain (IL-6, IL-11, CNTF, and CLC) first bind to the co-receptor (IL-6R, IL-11R, and CNTFR) through binding site I. The glycoprotein gp130 always interacts through binding site II and, depending on the cytokine, the third binding site (site III) is used for recruitment of LIFR, OSMR or a second gp130 molecule. Research has shown that a conserved FXXK motif (SEQ ID NO:12) at the core of site III is essential for all LIFR binding proteins for their interaction with LIFR. After recruiting the required receptors, these cytokines signal via activation of Jak/STAT (Janus kinase/signal transducer and activator of transcription) and MAPK (mitogen activated protein kinase) pathways.

A comprehensive list of many biological responses triggered by IL-6 type cytokines is outlined by Heinrich et al., and Nakashima & Taga. Among the many functions that these molecules can elicit, the inventors have shown that the IL-6 family of cytokines plays an important role in endogenously induced neuroprotection. Preconditioning with moderate oxidative stress (e.g., moderate bright light or mild hypoxia) is shown to induce changes in retinal tissue that protect photoreceptors from a subsequent dose of lethal oxidative stress. The inventors have shown that the IL-6 family of cytokines are strongly up regulated in response to preconditioning with bright cyclic light, and that these molecules are essential for the induced protection mediated by activation of LIFR: gp130 and its downstream signal, STAT3. In addition, a number of studies have shown that artificial injection of these cytokines protects photoreceptor cells in the retina from oxidative damage induced by light stress or inherited genetic mutations.

Therefore, the inventors hypothesized that creation of agonists that can mimic or promote the actions of these cytokines would prove very valuable in treating a number of disease states associated with gp130 and its ligands.

Oncostatin M (OSM) was originally isolated in 1986 from the growth media of phorbol 12-myristate 13-acetate (PMA)-treated U-937 histiocytic lymphoma cells; OSM was identified by its ability to inhibit the growth of melanoma cell lines. Later, it was shown to inhibit the growth of several other types of tumor cells including lung cancer cells, breast cancer cells, glioma cells and solid tissue tumor cells (Horn et al., 1990; Liu et al., 1997; and Halfter et al., 1998). However, growing evidence suggests that OSM acts on a wide variety of cells in vivo and elicits diverse biological responses involved in inflammation, neuroprotection, hematopoiesis, tissue remodeling and development.

Among its family members, OSM resembles Leukemia Inhibitory Factor (LIF) most closely both in structure and function. The gene encoding for OSM, located on human chromosome 22q12, is only 20 kb away from LIF, suggesting that these two genes evolved by gene duplication. In spite of the striking similarities, OSM differs from LIF in its receptor binding. While LIF first binds to leukemia inhibitory factor receptor (LIFR) and then recruits glycoprotein 130 (gp130) for its signal transduction, OSM first binds gp130 and then recruits LIFR. In addition, OSM can bind to gp130 and then recruit a unique receptor named Oncostatin M receptor (OSMR), thereby forming a new signaling complex.

While the native IL-6 family of cytokines can perform the desired function of activating the gp130 signaling cascade, new and improved activators that can be used at lower (i.e., less toxic) dosage levels and that are available in a more stable form when compared to native cytokines, are needed.

Therefore, there is a need in the art for new and improved methods of activating signal transducing receptors, and in particular gp130, that exhibit improvement when compared to native cytokines, and that overcome the disadvantages and defects of the prior art. It is to said compositions of modulating signal transducing receptors, as well as methods of producing and using same, that the presently disclosed and claimed inventive concept(s) is directed.

### BRIEF DESCRIPTION OF THE DRAWINGS

FIG. 1 illustrates crystal structures of LIF (PDB: 1EMR) (Panel A) and OSM (PDB: 1EVS) (Panel B) with their active sites and helices A, B, C and D identified. Both structures have an “up-up-down-down” topology with the N-terminus and C-terminus indicated. Also identified is the helical loop on OSM between its B and C helices. Panel C shows the alignment of OSM structure onto LIF based on the α-carbon trace; RMSD=4.342. Panel D depicts the three dimensional model for LIF in complex with LIFR and gp130 in presence of OSM overlaid on LIF. FXXK sequence of active site III is SEQ ID NO:12.

FIG. 2 illustrates the amino acid sequence of human OSM (SEQ ID NO:1). All alpha helical regions in OSM are highlighted in gray with the helices A (Y10-137; SEQ ID NO:2), B (G66-Q90; SEQ ID NO:3), C (E106-L131; SEQ ID NO:4) and D (A159-S185; SEQ ID NO:5) indicated. The cryptic thrombin cleavage site ‘AGR’ between helix C and helix D is highlighted in a box.

FIG. 3 depicts an SDS-PAGE analysis of human OSM (hOSM) with or without the ‘AGA’ modification after subjection to thrombin cleavage. Lane 1—hOSM; Lane 2—hOSM with AGA modification.

FIG. 4 depicts an SDS-PAGE analysis of the purified proteins LIF, OSM-WT, OSM-M1 and OSM-M2. 8 μg of purified protein was loaded into each lane.

FIG. 5 illustrates the amino acid sequences of wild type OSM (SEQ ID NO:1) and the mutant variants of OSM with truncated BC loops (OSM-M1, SEQ ID NO:6; OSM-M2, SEQ ID NO:7; and OSM-M3, SEQ ID NO:8). Shown in gray are the α-helices present in the secondary structure of OSM as identified in the crystal structure (PDB: 1EVS). Each of the helices A, B, C and D are identified (SEQ ID NOS:2-5, respectively), along with the BC loop region (SEQ ID NO:9 for the BC Loop of OSM-WT; SEQ ID NO:10 for BC Loop of OSM-M1; and SEQ ID NO:11 for BC Loop of OSM-M2). Also highlighted in the open box is the mutated thrombin cleavage site “AGA”.

FIG. 6 graphically illustrates that the modifications in the BC loop area of OSM did not induce a global change in the protein's structure. Panel A: Average of 3 CD spectra of the purified proteins plotted as molar ellipticity (θ) versus the wavelength. Panel B: Theoretical estimation of the secondary structural content for each protein using SELCON3, CDSSTR and CONTINNL. Values are presented as mean of estimations given by the three programs±SD.

FIG. 7 illustrates that Human Miller cells express LIFR and gp130, while A375 melanoma cells express OSMR and gp130 on their cell surface. Panel A: Activation of STAT3 in human retinal Müller cells in response to different doses of LIF and OSM. Panel B: Activation of STAT3 in human retinal Willer cells in response to different doses of LIF and OSM in the presence or absence of various doses of LIFOS (LIFR antagonist). Panel C: Activation of STAT3 in A375 melanoma cells in response to various doses of LIF and OSM.

FIG. 8 graphically illustrates activation of STAT3 and ERK in A375 melanoma cells in response to different doses of wild type (OSM-WT) and the mutant forms of OSM (OSM-M1 and OSM-M2), in Panel A. In Panels B and C, band intensities of phospho STAT3 (Panel B) and phospho ERK (Panel C) normalized against the band intensities of β-actin were plotted against the concentration of cytokines used for stimulation. Values are presented as mean±SE. n≧4. (*p<0.01, **p<0.001, compared to OSM-WT treatment at same dose). Shown for comparison is the normalized phospho STAT3 induced by LIF as estimated by the representative data shown in FIG. 7C.

FIG. 9 depicts activation of STAT3 and ERK in human Müller cells in response to different doses of wild type (OSM-WT) and the mutant forms of OSM (OSM-M1 and OSM-M2), in Panel A. In Panels B and C, band intensities of phospho STAT3 (Panel B) and phospho ERK (Panel C) normalized against the band intensities of β-actin were plotted against the concentration of cytokines used for stimulation. Values are presented as mean±SE. n≧4. (*p<0.05, **p<0.01, compared to OSM-WT treatment at same dose). Shown for comparison is the normalized phospho STAT3 induced by LIF as estimated by the representative data shown in FIG. 7A.

FIG. 10 demonstrates that the OSM with truncated BC loop still utilizes the FXXK motif to activate LIFR and OSMR. Activation of STAT3 in A375 melanoma and human Willer cells after stimulation with 1 ng/ml concentration of various forms of OSM containing either the wild type (FXXK; SEQ ID NO:12) or alanine substituted (AXXA; SEQ ID NO:13) active site III. 15 μg of total protein was loaded into each lane.

FIG. 11 illustrates a kinetic analysis of soluble LIFR and soluble gp130 interaction with LIF, OSM-WT, OSM-M1 or OSM-M2. Soluble LIFR (left panel) or soluble gp130 (right panel) at various concentrations were injected over an SPR sensor chip with immobilized ligand (LIF, OSM-WT, OSM-M1 or OSM-M2). Models are indicated by a smooth gray line overlaid over response curve traces.

FIG. 12 graphically depicts a kinetic analysis of soluble OSMR interaction with LIF, OSM-WT, OSM-M1 or OSM-M2. Neither wild type (OSM-WT) nor the mutant forms of OSM with truncated BC loops (OSM-M1 and OSM-M2) exhibit a direct affinity towards OSMR. Soluble OSMR at various concentrations were injected over an SPR sensor chip with immobilized ligands (LIF, OSM-WT, OSM-M1 or OSM-M2) at flow rates of 25 μl/min. Responses obtained were corrected for background signal using a control flow cell. Association and dissociation rates were derived by global analysis of the response curves fit to a 1:1 kinetic model using QDat software (BioLogic Software, Ltd. Knoxville Tenn. and Nomadics, Inc. Stillwater, Okla.) using 1:1 stoichiometry. Models are indicated by a smooth gray line overlaid over response curve traces.

FIG. 13 graphically depicts an ELISA analysis of soluble OSMR and soluble LIFR binding with LIF, OSM-WT, OSM-M1 and OSM-M2 or gp130 bound LIF (gp130:LIF), OSM-WT (gp130:OSM-WT), OSM-M1 (gp130:OSM-M1) and OSM-M2 (gp130:OSM-M2). Cytokines (LIF, OSM-WT, OSM-M1 or OSM-M2) immobilized on an ELISA plate in the absence (A,C) or presence (B,D) of gp130 were treated with various concentrations of soluble OSMR (A,B) or soluble LIFR (C,D). Equilibrium K_{D} values were estimated using a non-linear curve fitting to the binding data using GraphPad Prism (Graph Pad Software. LaJolla, Calif.). In addition, see Table 3.

FIG. 14 depicts A375 melanoma cell proliferation in the presence or absence of various doses of OSM-WT, OSM-M1 and OSM-M2 (Panels A, B and C, respectively). In Panel D, cell numbers on the 5th day of proliferation were normalized against the control cells and plotted for comparison. Values are presented as mean±SD. n=4 (*p<0.01, **p<0.001, compared to control treatment at same dose).

FIG. 15 graphically depicts that mutant OSM's with truncated BC loops (OSM-M1 and OSM-M2) were more potent than the wild-type OSM (OSM-WT) in protecting photoreceptor cells from light damage (LD). (A) Representative sections and (B) quantification of number of rows of photoreceptor nuclei in the outer nuclear layer (ONL) along the vertical meridian of the retinas treated with PBS (□), OSM-WT (), OSM-M1 (♦) or OSM-M2 (▴) and subjected to light damage (4000 lux for 4 hours). Quantification of photoreceptor nuclei layers from a normal retina (▪) is shown for comparison. n=3; value=mean SD (*p<0.05, vs. LD eyes, paired t-test). ONL—outer nuclear layer; INL—inner nuclear layer; GCL—ganglion cell layer).

### DETAILED DESCRIPTION OF THE PRESENTLY DISCLOSED AND CLAIMED INVENTIVE CONCEPT(S)

Before explaining at least one embodiment of the presently disclosed and claimed inventive concept(s) in detail by way of exemplary drawings, experimentation, results, and laboratory procedures, it is to be understood that the presently disclosed and claimed inventive concept(s) is not limited in its application to the details of construction and the arrangement of the components set forth in the following description or illustrated in the drawings, experimentation and/or results. The presently disclosed and claimed inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The terms “peptide”, “polypeptide” and “protein” are used herein to refer to a polymer of amino acid residues. The term “isolated peptide/polypeptide/protein” as used herein refers to a peptide/polypeptide/protein of cDNA, recombinant RNA, or synthetic origin or some combination thereof, which by virtue of its origin, or source of derivation, the “isolated peptide/polypeptide/protein”: (1) is not associated with peptides/polypeptides/proteins found in nature, (2) is free of other peptides/polypeptides/proteins from the same source, e.g., free of murine proteins, (3) is expressed by a cell from a different species, and/or (4) does not occur in nature.

The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby expressly incorporated by reference. An oligonucleotide can include a label for detection, if desired.

The term “selectively hybridize” referred to herein means to detectably and specifically bind. Polynucleotides, oligonucleotides and fragments thereof in accordance with the presently disclosed and claimed inventive concept(s) selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the presently disclosed and claimed inventive concept(s) and a nucleic acid sequence of interest will be at least 80%, and more typically with increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, such as at least 90 to 95 percent sequence identity, or at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the presently disclosed and claimed inventive concept(s). Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, such as at least 90 percent sequence identity, or at least 95 percent sequence identity, or at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of proteins/polypeptides are contemplated as being encompassed by the presently disclosed and claimed inventive concept(s), providing that the variations in the amino acid sequence maintain at least 75%, such as at least 80%, 90%, 95%, and 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of proteins/polypeptides can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the presently disclosed and claimed inventive concept(s).

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure©. Branden and J. Tooze, eds., Garland Publishing, New York, N.Y. (1991)); and Thornton et al. Nature 354:105 (1991), which are each incorporated herein by reference.

The term “gp130” as used herein refers to glycoprotein 130, a cell surface receptor that is expressed ubiquitously in the body. Activation of gp130 is essential for several physiological functions, including but not limited to, acute-phase response to injury and infection, fertility, metabolism, haematopoiesis, neuroprotection, anti-angiogenesis, and melanoma and tumor cell suppression. Gp130 is activated by a ligand from the IL-6 family of cytokines, including but not limited to, IL-6, IL-11, leukemia inhibitory factor (LIF), Oncostatin M (OSM), ciliary neurotrophic factor (CNTF), cardiotrophin-1 (CT-1) and cardiotrophin-like cytokine (CLC). Activation of gp130 signaling may be direct, i.e. activation may be triggered by binding of the ligand directly to gp130 (i.e., IL-6 or IL-11, which result in gp130-homodimerization). Activation of gp130 signaling may also be indirect by binding of the ligand to another cell surface receptor, which forms a complex with gp130, thereby activating it. LIF, CT-1, CNTF, OSM and CLC form heterodimers of gp130 and LIFR, whereas OSM may also form a heterodimer of gp130 and OSMR. Therefore, LIF, CT-1, CNTF, OSM and CLC may activate gp130 signaling directly, by binding gp130 first, or indirectly, by binding LIFR/OSMR and then recruiting gp130 to the complex. The ligands of the IL-6 cytokine family trigger the JAK/STAT pathway, the first event of which is the ligand-induced homo- or hetero-dimerization of signal-transducing receptor subunits. All IL-6-type cytokines recruit gp130 to their receptor complexes. They either signal via gp130 alone or in combination with LIFR or OSMR, which are all able to activate Jaks and to recruit STAT proteins.

The term “modulator” as used herein will be understood to refer to a variant of a ligand that exhibits an increased activity when compared to a wild type ligand. The modulator may exhibit an actual increase in the activity of the wild type ligand, or the modulator may exhibit an increase in potency when compared to wild type (i.e., the modulator obtains the same effect as the wild type but at a lower dosage level), or the modulator may exhibit an increase in stability when compared to wild type (thereby increasing the duration of the activity of the modulator when compared to wild type). The increase in stability may be achieved by mutating at least one protease cleavage site in the amino acid sequence, thereby rendering the modulator resistant to protease cleavage.

The terms “ligand variant”, “variant of a ligand”, “derivative of a ligand” and “ligand derivative” are used interchangeably herein and will be understood to refer to a polypeptide molecule that is (a) a mutagenized form of a native ligand, or (b) a polypeptide produced through recombination that has at least one mutation when compared to a native ligand; however, said polypeptide molecule still retains the desired activity of an ability to bind to a receptor and thereby directly or indirectly activate a signaling pathway.

The term “agonist” as used herein will be understood to refer to a type of ligand, such as, but not limited to, a drug or hormone, that binds to receptors and thereby alters the proportion thereof that are in an active form, resulting in a biological response. An agonist of particular interest herein is one which mimics one or more (e.g. all) of the biological properties of the naturally occurring ligand (i.e., IL-6 cytokine family member). In preferred embodiments, the agonist has a biological property of the naturally-occurring ligand (i.e., IL-6 cytokine family member) which is the same activity or an increased activity when compared to the naturally-occurring ligand, as described herein above.

The term “effective amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the presently disclosed and claimed inventive concept(s). The therapeutic effect may include, for example but not by way of limitation, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that a patient in need of treatment is treated or given another drug for the disease or condition in conjunction with the pharmaceutical compositions of the presently disclosed and claimed inventive concept(s). This concurrent therapy can be sequential therapy where the patient is treated first with one drug and then the other, or the two drugs can be given simultaneously.

The terms “administration” and “administering”, as used herein will be understood to include all routes of administration known in the art, including but not limited to, oral, topical, transdermal, parenteral, subcutaneous, intranasal, mucosal, intramuscular, intraperitoneal, intravitreal and intravenous routes, including both local and systemic applications. In addition, the methods of administration may be designed to provide delayed or controlled release using formulation techniques which are well known in the art.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

Certain pharmaceutical compositions prepared in accordance with the presently disclosed and claimed inventive concept(s) are single unit dosage forms suitable for oral, mucosal (e.g., nasal, sublingual, vaginal, buccal, or rectal), parenteral (e.g., subcutaneous, intravenous, bolus injection, intramuscular, or intraarterial), intravitreal, or transdermal administration to a patient. Examples of dosage forms include, but are not limited to, tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation of the presently disclosed and claimed inventive concept(s) should suit the mode of administration. For example, oral administration requires enteric coatings to protect the agents of the presently disclosed and claimed inventive concept(s) from degradation within the gastrointestinal tract. In another example, the agents of the presently disclosed and claimed inventive concept(s) may be administered in a liposomal formulation to shield the agents from degradative enzymes, facilitate transport in circulatory system, and effect delivery across cell membranes to intracellular sites.

The composition, shape, and type of dosage forms of the pharmaceutical compositions of the presently disclosed and claimed inventive concept(s) will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by the inventive concept(s) will vary from one another and will be readily apparent to those skilled in the art. See, e.g., Remington: The Science and Practice of Pharmacy, Alfonso R. Gennaro, editor, 20th ed. Lippincott Williams & Wilkins: Philadelphia, Pa., 2000.

By “biologically active” is meant the ability to modify the physiological system of an organism. A molecule can be biologically active through its own functionalities, or may be biologically active based on its ability to activate or inhibit molecules having their own biological activity.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” as used herein includes human and veterinary subjects. “Mammal” for purposes of treatment refers to any animal classified as a mammal, including human, domestic and farm animals, nonhuman primates, zoo, sports, or pet animals, such as dogs, horses, cats, cows, etc., and any other animal that has mammary tissue.

“Treatment” refers to both therapeutic treatment and prophylactic or preventative measures. Those in need of treatment include, but are not limited to, individuals already having a particular condition or disorder as well as individuals who are at risk of acquiring a particular condition or disorder (e.g., those needing prophylactic/preventative measures). The term “treating” refers to administering an agent to a patient for therapeutic and/or prophylactic/preventative purposes.

Administering a therapeutically effective amount or prophylactically effective amount is intended to provide a therapeutic benefit in the treatment, prevention, or management of a disease and/or disorder. The specific amount that is therapeutically effective can be readily determined by the ordinary medical practitioner, and can vary depending on factors known in the art, such as the type of disease/disorder, the patient's history and age, the stage of disease/disorder, and the co-administration of other agents.

A “therapeutic agent” refers to an agent that may be administered in vivo to bring about a therapeutic and/or prophylactic/preventative effect.

A “disorder” is any condition that would benefit from treatment with the polypeptide. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of include but are not limited to, melanoma, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

The presently disclosed and claimed inventive concept(s) is related to compositions comprising at least one functionally active modulator for gp130. The agonist/modulator may function by binding to gp130 and causing gp130 to homo-dimerize with another gp130 molecule or hetero-dimerize with another receptor, such as but not limited to, leukemia inhibitory factor (LIFR) or Oncostatin M receptor (OSMR). Said binding and dimerization result in activation of gp130, whereby the activation of gp130 is increased when compared to activation of gp130 by a wild type molecule. Said increase in activation of gp130 will also result in increased activation of downstream signaling pathways, including but not limited to STAT3 and MAPK pathways. In addition, said increase can also activate several functions inside the body, including but not limited to, acute-phase responses to injury and infection, fertility, metabolism, haematopoiesis, neuroprotection, anti-angiogenesis, and melanoma and tumor cell suppression.

Said “increase in activation of gp130” may be the result of an actual increase in the activation itself, or may be an increase in the potency of the modulator when compared to a wild type ligand (i.e., the modulator obtains the same effect as the wild type ligand but at a lower dosage level), or may be an increase in the stability of the modulator when compared to the wild type ligand, thereby increasing the duration of the activation of gp130. The increase in stability may be achieved by mutating at least one protease cleavage site in the amino acid sequence of the wild type ligand, thereby rendering the modulator resistant to protease cleavage.

In one embodiment, the compositions comprise an agonist that mimics or promotes the action of at least one member of the IL-6 family of cytokines, such as but not limited to, a variant of a native ligand for gp130. The agonist may have a similar structure to the at least one member of the IL-6 family of cytokines, whereby the agonist has at least one amino acid sequence substitution, addition and/or deletion when compared to the at least one member of the IL-6 family of cytokines, i.e., a modified version of a native IL-6 cytokine family member (including but not limited to, IL-6, IL-11, leukemia inhibitory factor (LIF), Oncostatin M (OSM), ciliary neurotrophic factor (CNTF), cardiotrophin-1 (CT-1) and cardiotrophin-like cytokine (CLC)).

In one embodiment, the functionally active modulator for gp130 is an agonist that is a variant of Oncostatin M (OSM). Activation of gp130 by the variant is increased when compared to activation of gp130 by wild type OSM. The OSM variant has at least one amino acid sequence truncation, deletion or substitution in a BC loop thereof when compared to wild type OSM, and said modification to the BC loop decreases steric hindrance and increases affinity of the variant for leukemia inhibitory factor (LIFR) and/or Oncostatin M receptor (OSMR) when compared to wild type OSM.

In certain embodiments, the wild type OSM may comprise the amino acid sequence of SEQ ID NO:1, and the variant may have at least one amino acid sequence truncation, deletion or substitution in SEQ ID NO:9 (i.e., the BC loop sequence) thereof. Depending on the amount of the truncation and/or deletion in SEQ ID NO:9, the amino acid sequence of the OSM variant may further comprise one or more amino acid additions to induce flexibility in the loop region and to minimize the impact of the modification on the overall structure of the OSM variant.

The OSM variant may comprise an amino acid sequence selected from the group consisting of SEQ ID NOS:6-8. As mentioned above, when the OSM variant comprises SEQ ID NO:8, the amino acid sequence will further comprise one or more amino acid additions to induce flexibility in the loop region and to minimize the impact of the modification on the overall structure of the OSM variant.

The OSM variant may comprise an amino acid sequence that comprises (a) at least one amino acid sequence substitution, addition and/or deletion when compared to SEQ ID NO:1; and (b) comprises the five motifs of SEQ ID NOS:2-5 and 12. In another embodiment, the OSM variant may comprise an amino acid sequence that is at least 90% identical to SEQ ID NO:1. In yet another embodiment, the OSM variant may comprise an amino acid sequence encoded by a nucleotide sequence capable of hybridizing to a complement of a nucleic acid sequence encoding SEQ ID NO:1 under stringent hybridization conditions, as discussed in detail herein. In a further embodiment, the compositions may comprise any combination of the embodiments described herein above.

The presently disclosed and claimed inventive concept(s) is also related to isolated and purified nucleic acid segments encoding at least one composition described herein above. In one embodiment, the isolated nucleic acid segments of the presently disclosed and claimed inventive concept(s) may encode an agonist that is a variant of Oncostatin M (OSM), whereby the variant exhibits increased activation of gp130 when compared to native OSM, and wherein the OSM variant has at least one amino acid sequence truncation, deletion or substitution in a BC loop thereof when compared to wild type OSM. For example, the isolated nucleic acid segments of the presently disclosed and claimed inventive concept(s) may encode an amino acid sequence of at least one of SEQ ID NOS:6-8. In another embodiment, the isolated nucleic acid segments may comprise a nucleic acid sequence encoding an amino acid sequence that is at least 90% identical to SEQ ID NO:1. In yet another embodiment, the isolated nucleic acid segments may comprise a nucleic acid sequence capable of hybridizing to a complement of a nucleic acid sequence encoding SEQ ID NO:1 under stringent hybridization conditions, as discussed in detail herein. In another embodiment, the isolated nucleic acid segments may comprise a nucleic acid sequence encoding an amino acid sequence that comprises at least one amino acid sequence substitution, addition and/or deletion when compared to SEQ ID NO:1 and comprises the five motifs of SEQ ID NOS:2-5 and 12. In yet another embodiment, the isolated nucleic acid segments comprise a nucleic acid segment encoding an amino acid sequence that comprises at least one amino acid sequence truncation, deletion or substitution in SEQ ID NO:1, and wherein said at least one amino acid sequence truncation, deletion or substitution occurs in SEQ ID NO:9 of SEQ ID NO:1. In yet another embodiment, the isolated nucleic acid segments may comprise a nucleic acid sequence encoding an amino acid sequence that comprises at least one amino acid sequence substitution, addition and/or deletion when compared to SEQ ID NO:1, wherein said at least one substitution, addition and/or deletion occurs in a protease cleavage site of SEQ ID NO:1, whereby the resultant polypeptide is resistant to protease cleavage.

One non-limiting example of stringent hybridization conditions that may be utilized in accordance with the presently disclosed and claimed inventive concept(s) includes 6× sodium chloride/sodium citrate (SSC) at about 45° C., followed by 0.2×SSC, 0.1% SDS at 50-65° C.

The presently disclosed and claimed inventive concept(s) also includes a recombinant vector comprising at least one of the nucleic acid segments described herein above. Further, the presently disclosed and claimed inventive concept(s) also includes a recombinant host cell comprising the recombinant vector.

The presently disclosed and claimed inventive concept(s) also includes a pharmaceutical composition comprising said functionally active modulator for gp130 (or a nucleic acid segment encoding same). The pharmaceutical composition may further comprise at least one additional agent, as described in detail herein. The presently disclosed and claimed inventive concept(s) also includes a pharmaceutical composition comprising a therapeutically effective amount of at least one of the compositions described herein in combination with a pharmaceutically acceptable carrier. As used herein, a “pharmaceutically acceptable carrier” is a pharmaceutically acceptable solvent, suspending agent or vehicle for delivering the compounds of the present invention to the human or animal. The carrier may be liquid or solid and is selected with the planned manner of administration in mind. Examples of pharmaceutically acceptable carriers that may be utilized in accordance with the present invention include, but are not limited to, PEG, liposomes, hydrogels, ethanol, DMSO, aqueous buffers, oils, and combinations thereof.

The presently disclosed and claimed inventive concept(s) also includes a method of identifying a functionally active modulator of gp130. The method comprises providing the sequence of at least one native IL-6 cytokine family member and modifying the sequence by adding, deleting and/or substituting at least one amino acid thereof, then determining if the protein produced therefrom forms a more stable complex with gp130 and thereby increases the activation of gp130 when compared to the native IL-6 cytokine family member.

In one embodiment, the method includes providing SEQ ID NO:1, and modifying at least a portion of SEQ ID NO:9 therein, then determining if the polypeptide produced therefrom forms a more stable complex with gp130 and/or exhibits increased affinity for LIFR and/or OSMR when compared to wild type OSM.

The presently disclosed and claimed inventive concept(s) is also directed to a method of producing a functionally active modulator of gp130. The method includes providing a host cell encoding one of the compositions described herein above, and culturing the host cell under conditions that allow for production of the at least one functionally active modulator of gp130 (i.e., the ligand variant).

The presently disclosed and claimed inventive concept(s) also includes a method of activating at least one gp130 signaling cascade. Said method comprises providing at least one cell having gp130 expressed on a surface thereof, and providing a composition comprising a functionally active modulator of gp130 as described herein above. The composition is administered to the cell in an effective amount, whereby the composition binds to gp130 and causes gp130 to homo-dimerize or heterodimerize with LIFR or OSMR on the surface of the cell, whereby the binding and dimerization activates at least one gp130 signaling cascade. The signaling cascade may include, but is not limited to Janus kinase/signal transducer and activator of transcription (Jak/STAT) and mitogen activated protein kinase (MAPK).

The presently disclosed and claimed inventive concept(s) also includes a method of activating at least one gp130 signaling cascade in a patient. In said method, a therapeutically effective amount of the pharmaceutical composition described herein above is administered to the patient, whereby the composition binds to the gp130 and causes gp130 to homodimerize or heterodimerize with LIFR or OSMR on the surface of the cell, whereby the binding and dimerization activates at least one gp130 signaling cascade. The signaling cascade may include, but is not limited to Janus kinase/signal transducer and activator of transcription (Jak/STAT) and mitogen activated protein kinase (MAPK).

Activation of the at least one gp130 signaling cascade may affect one or more of the following in the patient: acute-phase response to injury and infection, fertility, metabolism, haematopoiesis, neuroprotection, anti-angiogenesis, and melanoma and tumor cell suppression.

The presently disclosed and claimed inventive concept(s) is further directed to a method for providing neuroprotection to a patient in need thereof. The method includes administering to the patient a therapeutically effective amount of one of the pharmaceutical compositions described herein above.

The presently disclosed and claimed inventive concept(s) is further directed to a method for providing retinal neuroprotection to a patient in need thereof. The method includes administering to at least one eye of the patient a therapeutically effective amount of one of the pharmaceutical compositions described herein above. In one embodiment, the pharmaceutical composition is intravitreally injected into the at least one eye. In another embodiment, the pharmaceutical composition is topically applied. In said method, the neuroprotective effect may diminish, or protect the patient from, at least one of neuronal damage caused by exposure to oxidative stress, neuronal damage caused by light stress, and retinal degeneration induced by inherited genetic mutation.

The pharmaceutical composition may be administered before onset of neuronal damage, or administered therapeutically after onset of neuronal damage.

The presently disclosed and claimed inventive concept(s) is yet further directed to a method of treating, preventing, or reducing the occurrence of a disorder in a mammal in need of such treatment. The method includes administering a therapeutically effective amount of one of the pharmaceutical compositions described herein above. The disorder may be selected from the group consisting of age related degenerations, progressive degenerations, acute pancreatitis, Alzheimer's disease, generically inherited mutations, obesity, diabetes, insulin resistance, glucose intolerance, dyslipidemia, hypertension, hypercholesterolemia, cancer, melanoma, inflammation and inflammatory disorders including but not limited to inflammatory arthropathy, gout, rheumatoid arthritis, osteoarthritis, and inflammatory vascular diseases (both peripheral and central), injury, infection, infertility, haematopoietic disorders, angiogenetic disorders, and combinations thereof.

Delivery of the agents of the presently disclosed and claimed inventive concept(s) into a patient can either be direct, i.e., the patient is directly exposed to an agent of the presently disclosed and claimed inventive concept(s) or agent-carrying vector, or indirect, i.e., cells are first transformed with the nucleic acid sequences encoding an agent of the presently disclosed and claimed inventive concept(s) in vitro, then transplanted into the patient for cell replacement therapy. These two approaches are known as in vivo and ex vivo therapy, respectively.

Regarding the use of the pharmaceutical compositions of the presently disclosed and claimed inventive concept(s) to treat the various disorders listed herein above, member of the IL-6 family of cytokines, including OSM, have been shown to act on a wide variety of cells and elicit diverse overlapping biological responses such as but not limited to, inflammation, neuroprotection, haematopoiesis and development. Since OSM shows therapeutic potential in each of these conditions, the OSM variants of the presently disclosed and claimed inventive concept(s) will not only find application in these same therapeutic uses, but said variants will have greater therapeutic potential than OSM. Said OSM variants will exhibit greater activity at lower concentrations when compared to wild type OSM. A brief summary of the knowledge regarding uses of OSM in treatment of various disorders is provided herein below.

Inflammation refers to a complex set of mechanisms by which tissues respond to injury and infection. The initial signs of swelling, pain and heat are characteristics of the initial phase of inflammation, termed as acute inflammatory response. This is characterized by increased blood flow, increase in permeability of the surrounding capillaries and infiltration of white blood cells, predominantly neutrophils. In case of severe damage, this reaction is followed by the chronic inflammatory response where the affected tissue is infiltrated by lymphocytes, macrophages, and mast cells. Substantial tissue remodeling can occur during this phase which may lead to complete restoration of normal tissue architecture or a scar formation. This complex chain of events is regulated by an array of mediators, which includes cytokines, the extracellular matrix, and adhesion molecules.

Activated T cells and monocytes at the site of injury/infection secrete Oncostatin M (Grenier et al., 1999; and Boniface et al., 2007), which in turn stimulates endothelial cells to secrete IL-6 in the blood stream (Brown et al., 1991). IL-6 then stimulates the liver to secrete acute phase proteins (APPs) into circulation. APPs are essential for controlling body homeostasis and regulate the inflammatory response. While other IL-6 family cytokines like LIF, CNTF and IL-11 also stimulate the release of APPs from the liver, IL-6 was found to be the primary inducer of APPs in vivo. In addition to the APPs, OSM and IL-6 are shown to modulate the expression of other cytokines and chemokines involved in inflammation e.g., IL-1, IL-8, granulocyte macrophage-colony stimulating factor (GM-CSF), growth related oncogenes α and β. Also, OSM induces prolonged expression of P-selectin and E-selectin in endothelial cells, which modulate leukocyte adhesion and extravasation. This is an important phenomenon involved in wound healing.

During the repair process, remodeling of extracellular matrix plays an important role in healing the damaged tissue. Matrix metalloproteinases (MMPs) are involved in extracellular matrix breakdown while tissue inhibitors of metalloproteinases (TIMPs) inhibit the action of MMPs. OSM plays a crucial role in this process by modulating the expression of TIMP-1 and MMP-1, MMP-3 and MMP-9 in fibroblasts at the wound site.

Besides acute phase reactions, IL-6 family cytokines are also associated with several acute and chronic inflammatory diseases, such as but not limited to, rheumatoid arthritis, acute pancreatitis, and Alzheimer's disease. In patients suffering from rheumatoid arthritis, elevated levels of IL-6, IL-11, LIF and OSM have been found in the synovial fluids and the serum, and the levels of these cytokines were shown to correlate with disease severity. Research has shown that these cytokines induce bone remodeling, stimulate cartilage degradation and induce osteoblast proliferation. Injection of anti-IL-6 receptor monoclonal antibody and LIF antagonists were shown to ameliorate inflammatory reactions in these inflammatory models.

IL-6 family cytokines LIF, CNTF, OSM and CT-1, all of which signal through heterodimerization of gp130 with LIFR, exhibit a wide range of roles in both the developing and mature nervous system. They play a vital role in modulating the differentiation of neuronal cells and promote their survival under stress conditions. While most of these cytokines are expressed in various parts across the body, CNTF is found exclusively in the nervous system.

Initial studies using cytokine knockout models revealed no abnormalities in development, suggesting that these cytokines are not essential for neuronal development. However, CNTFRα and LIFR knockout mice died within 24 h of birth and had a marked reduction in numbers of spinal motor neurons, indicating that these cytokines in fact play an important role in neuronal survival and the overlapping signals executed by IL-6 family cytokines compensate for the loss of others. Although neurons and astrocytes are known to express LIF, CNTF, and OSM, glial cells are considered to be the major producers of these cytokines, and this expression is up regulated upon injury or stress by an as yet unknown mechanism. In vivo the neurons and astrocytes are closely associated with the glial cells thus exposing them to high concentrations of glial-derived neurotrophic factors and cytokines upon ischemic or excitotoxic injuries.

Excitotoxic pathways initiated after excessive glutamate release have been implicated in traumatic spinal cord injury, stroke and some chronic age-related neurodegenerative diseases like Alzheimer's disease. OSM was shown to significantly attenuate the neuronal cell death induced by a similar excitotoxic injury triggered by N-methyl-D-aspartate (NMDA), an analogue of glutamate, both in vitro and in vivo. Similar observations were made for CNTF. Treatment with exogenous CNTF was shown to protect the neurons after a CNS injury induced by both excitotoxic stimulation and by degenerative diseases like multiple sclerosis (MS) and Huntington's disease. Delivery of LIF, CNTF and OSM has been demonstrated to be neuroprotective in a number of other in vitro and in vivo models also. In the eye, studies by La Vail et al. have shown that multiple neurotrophic factors including CNTF rescue photoreceptors from damaging effects of constant light and retinal degenerations induced by inherited genetic mutations. Recent work by the inventors (Ueki et al., 2008) has shown that LIF, another IL-6 family member also protects the photoreceptors from oxidative stress induced by severe bright light. Later, knock out studies by Joly et al. (2008) have shown that endogenous LIF extends the life span of retinal photoreceptors in a mouse with degenerating retina induced by genetic mutations. In addition to these, studies by Rattner et al. (2008) have shown that the retina responds to severe light stress by up regulating the expression OSMR, suggesting a possible role of neuroprotection by OSM. Together, all these results clearly suggest that IL-6 family cytokines play an important role in protecting the neuronal cells from oxidative stress induced by injury or inherited genetic mutations. Separate studies have shown that preconditioning the eyes with bright cyclic light or with hypoxia also help the photoreceptors survive subsequent doses of severe oxidative stress. However, the molecules involved in this induced protection remain poorly characterized.

Hematopoiesis can be broadly defined as the regulation of the concentrations of cellular components in blood. In a healthy adult, approximately 10¹¹-10¹² new blood cells are produced daily in order to maintain the steady state levels. All of these cellular blood components are derived from hematopoietic stem cells (HSCs), which reside mainly in the bone marrow. These stem cells can proliferate and differentiate leading to the production of one or more specific types of blood cells. A number of factors control this process of proliferation and differentiation with great precision and regulate the production of blood cells.

While erythropoietin (Epo) and granulocyte-macrophage colony stimulating factor (GM-CSF) are the primary mediators of this regulation, IL-6 family cytokines also play an important role. mRNA levels for IL-6, IL-11, OSM and LIF are found to be abundant in hematopoietic tissues such as bone marrow, thymus and spleen. These cytokines, in concert with IL-3, are shown to regulate the proliferation of pluripotent hematopoietic progenitor cells by controlling their entry and exit from the cell cycle. Also, intravenous administration of LIF, OSM, IL-6 and IL-11 were all shown to result in dramatic increases in megakaryocyte and platelet numbers in the blood. In addition, IL-6 family members are also shown to inhibit the differentiation of macrophages and several myeloid leukemia cells, indicating that these cytokines play an important role in final maturation of the hematopoietic cells.

Oncostatin M was originally identified by virtue of its ability to suppress tumor cells. It was originally recognized in 1986 by its ability to inhibit the proliferation of A375 melanoma cells. Later, it was shown to inhibit the growth of several other types of tumor cells including lung cancer cells, breast cancer cells, glioma cells and solid tissue tumor cells. LIF, another IL-6 family member closely related to OSM, was not able to display a similar ability in suppressing tumor cells, suggesting that OSM executes these functions by recruiting its unique receptor OSMR. In agreement with these observations, more recent studies by Lacreusette et al. (2007) have shown that melanoma cell progression towards an OSM resistant metastatic state is accompanied by silencing of the OSMR gene. Thus, in addition to designing novel agonists of OSM, preventing the alteration of promoter region of OSMR can be a potential avenue to suppress the proliferation of tumor cells in vivo. Besides tumor cells, OSM also inhibits the proliferation of normal mammary and breast epithelial cells. In contrast, OSM stimulates the growth of AIDS related Kaposi's sarcoma cells and the normal dermal fibroblasts via mitogen-activated protein kinase (MAPK)-dependent pathway.

Increased low density lipoprotein-cholesterol (LDL-c) levels in plasma is a widely recognized risk factor for atherosclerosis and an important underlying cause for a number of cardiovascular diseases. The LDL receptor (LDLR) plays a pivotal role in the control of plasma cholesterol levels since more than 70% of the LDL-c in circulation is removed by LDLR-mediated endocytosis. Therefore, the regulation of liver LDLR expression has been considered a key mechanism by which therapeutic agents could interfere with the development of atherosclerosis.

Over the past three decades, statins (e.g., Rosuvastatin (Crestor®), Atorvastatin (Lipitor®), Lovastatin (Mevacor®)) have been extensively studied and applied in the clinical setting to serve as cholesterol depleting agents. They lower cholesterol levels by inhibiting the enzyme HMG-CoA reductase, which is the rate-limiting enzyme involved in cholesterol synthesis. Inhibition of this enzyme in the liver not only decreases cholesterol synthesis, but also increases synthesis of LDL receptors, leading to an increase in clearance of LDL-c from the bloodstream. In addition to these, LDLR expression levels were also shown to be regulated by several growth factors and cytokines. However, among these, OSM was shown to have the most pronounced effect in increasing the levels of LDLR on liver. When administered intraperitoneally, OSM was shown to induce rapid upregulation of LDLR expression in liver and this upregulation was sustained for 24 hrs. In addition, when tested in combination with the commercially available statins, OSM was found to show an additive effect. Further investigation showed that OSM regulates the LDLR expression through a separate statin independent mechanism. Clinical studies are under way to use OSM as a potential therapeutic agent to regulate cholesterol levels either independently or in combination with the currently available statins. See, e.g., Kong et al., 2005; Liu et al., 2003; and Zhang et al., 2003.

Obesity and its related cluster of pathophysiologic conditions, including but not limited to, insulin resistance, glucose intolerance, dyslipidemia, hypertension, and diabetes, are recognized as growing threats to world health. Recent research has focused on the role that gp130 receptor ligands may play as therapeutic targets in obesity. In exercising rats, hypothalamic insulin sensitivity was increased in an IL-6-dependent manner, and IL-6 has been shown to activate AMPK in both skeletal muscle and adipose tissue. Consistent with activation of AMPK, IL-6 has also been shown to increase fat oxidation in vitro, ex vivo, and in humans in vivo. Ciliary neurotrophic factor (CNTF) has been shown to act both centrally and peripherally and to mimic the biologic actions of the appetite control hormone leptin. CNTF has been shown to induce weight loss and improve glucose tolerance in humans and rodents; CNTF signaling through gp130 was shown to increase fatty-acid oxidation and reduce insulin resistance in skeletal muscle by activating AMP-activated protein kinase (AMPK), independent of signaling through the brain. CNTF treatment has demonstrated to be effective in the reduction of body weight, by promoting the inhibition of food intake and the activation of the energy expenditure, together with an improvement of insulin sensitivity. In addition, a human recombinant modified form of CNTF has been developed and clinically tested as an anti-obesity drug; while the results were promising, the efficacy of the modified form of CNTF appears limited, since patients treated with high doses of the drug reported side effects. Recent results showing potent peripheral effects of gp130 ligands in increasing lipid oxidation, activating AMPK, preventing lipid-induced inflammation, and upregulating genes associated with oxidative phosphorylation suggests that gp130 ligands may prove to be an important part of a therapeutic strategy to treat obesity. See, e.g. Febbraio, 2007; Watt et al., 2006; Matthews et al., 2008; Gloaguen et al., 1997; Marcos-Gomez et al., 2008; and Lambert et al., 2001.

Examples are provided hereinbelow. However, the present invention is to be understood to not be limited in its application to the specific experimentation, results and laboratory procedures. Rather, the Examples are simply provided as one of various embodiments and are meant to be exemplary, not exhaustive.

### Example 1

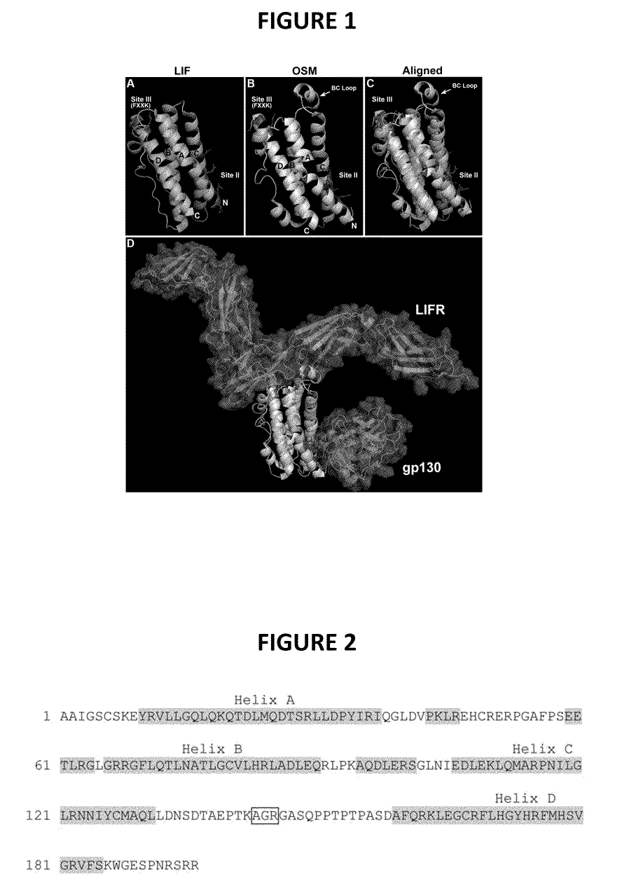

The present Example is related to identifying the structural features on OSM that result in its unique ability to bind OSMR and the features that result in its higher affinity towards gp130 than towards LIFR or OSMR. Based on the structural alignments, a helical loop on OSM has been identified between its B and C helices that is unique to OSM and not found on LIF or any other IL-6 cytokine family member (FIG. 1). Using wild type and mutant OSM molecules that have shortened BC loops, it is shown in this Example that the loop presents a steric hindrance for LIFR and OSMR, thus lowering the affinity for either receptor. Cytokines with deletions in the BC loop were able to activate LIFR:gp130 and OSMR:gp130 receptor complexes at 3 fold lower concentrations than the native OSM. Kinetic and equilibrium binding analysis of the ligand-receptor interactions show that improved activation is a consequence of increased affinity towards LIFR and OSMR without altering the affinity for gp130. Together, these results demonstrate that the BC loop modulates OSM's affinity towards LIFR and OSMR by presenting a steric hindrance for their interaction. This Example further demonstrates that the BC loop does not play a role in OSM's unique ability to bind OSMR.

Materials and Methods of Example 1

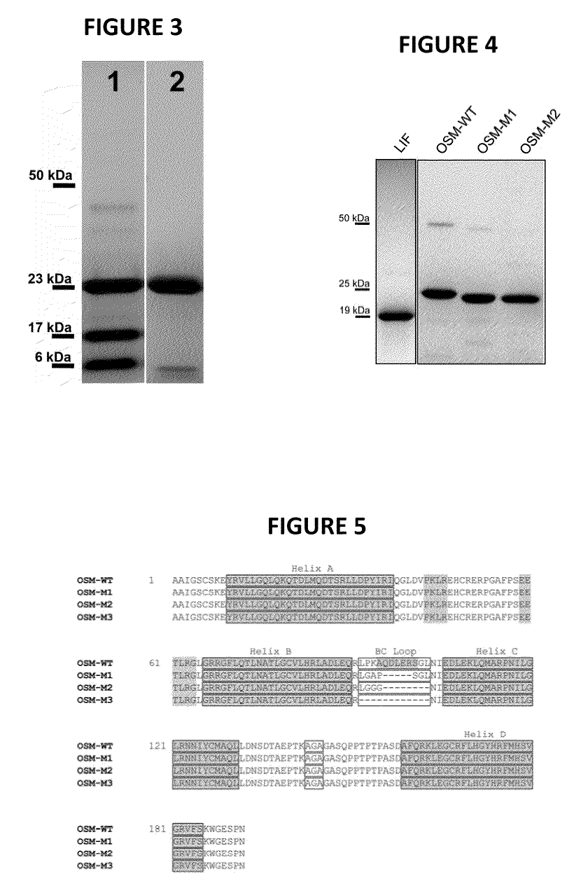

Protein Design: cDNA for hOSM was obtained from Invitrogen (Invitrogen, Carlsbad, Calif.), while the cDNA for hLIF and LIF05 were kindly donated by Dr. John K. Heath, University of Birmingham, UK. The gene encoding mature OSM was amplified using PCR with a FLAG tag introduced at its N-terminus. The gene was then cloned into a pGEX-2T vector for protein expression as a GST-FLAG fusion protein with a thrombin cleavage site between the GST and FLAG tags. During the course of purification, it was observed that the native human OSM contained a cryptic thrombin cleavage site ‘AGR’ between its C and D helices (FIG. 2). As expected, when this fusion protein was subjected to thrombin cleavage on a glutathione SEPHAROSE® 4B column, it resulted in the elution of two new fragments of sizes ˜17 kDa and ˜6 kDa in addition to the native OSM, which is ˜23 kDa (FIG. 3, lane 1). To facilitate recombinant protein purification and to increase protein stability in vivo, mutations were induced at the DNA level using the QUICKCHANGE® site directed mutagenesis kit (Stratagene, La Jolla, Calif.) to replace ‘AGR’ with ‘AGA’ (SEQ ID NO:28). This modification resulted in OSM that was resistant to thrombin cleavage (FIG. 3, lane 2). The ‘AGA’ modification did not alter OSM's functional activity on Müller cells (data not shown). This was expected, since the modification is located in a flexible loop region away from the receptor binding sites. From this point forward, this ‘AGA’ modified human OSM will be referred to as the wild-type OSM (OSM-WT). Recombinant proteins with modifications in the BC loop were made using the ‘AGA’ modified human OSM as the starting template. Therefore all recombinant OSM proteins expressed lack the thrombin cleavage site. Mutations and/or deletions of codons in the BC loop region were performed using QUICKCHANGE® mutagenesis kit (Stratagene, La Jolla, Calif.). Mutation of the FXXK motif (SEQ ID NO:12) on wild type and BC loop mutant OSM molecules to AXXA (SEQ ID NO:13) was also carried out using QUICKCHANGE® mutagenesis kit (Stratagene, La Jolla, Calif.) (See Table 1 for list of primers used).

Expression and purification of proteins: plasmids encoding wild-type or the mutant OSMs were transfected into Escherichia coli (E. coli) JM109 strain for protein expression. Cultures containing the inoculums were grown in LB plus ampicillin (100 μg/ml) at 37° C. and 300 rpm until they reached midlog phase (A₆₀₀=0.6). Isopropyl β-D-1-thyogalactopyranoside (IPTG) was then added to the culture to a final concentration of 0.1 mM, and induction was carried out for additional 3 hours at room temperature. Intracellular fusion protein was recovered from cell extracts by affinity binding to a slurry of glutathione-SEPHAROSE® 4B beads (GE Healthcare, Uppsala, Sweden). Washes were carried out as described by the manufacturer's protocol. Isolation of the FLAG tagged proteins was achieved by cleavage of the GST tag with human thrombin (Amersham Biosciences, Piscataway, N.J.) in 1×PBS (pH 7.3) overnight at room temperature. Following cleavage, the elution containing wild type or mutant OSM was pooled with additional 4 batch washes (1×PBS, pH 7.3). SDS-PAGE analysis of eluted proteins revealed that the E. coli expressed high amounts of wild-type, M1 and M2 versions of OSM. The M3 mutant version of OSM was not expressible in bacteria. This could be a result of structural instability in the M3 mutant version of OSM. Cleaved proteins were further purified by fast protein liquid chromatography (FPLC) using a MONO-Q® anionic exchange column (Amersham Biosciences, Piscataway, N.J.). Elution was carried out with a linear gradient of 0-1 M NaCl in 20 mM Tris buffer (pH 8.0). Eluted fractions were analyzed using SDS-PAGE. Fractions containing enriched protein were pooled and concentrated by ultrafiltration (Millipore Corporation, Billerica, Mass.). Identity of the proteins was confirmed by mass spectrometry and purities were >90%, as evaluated by Coomassie staining of the purified proteins run on a 4-20% gradient polyacrylamide gel (FIG. 4). Concentration of the purified recombinant proteins was estimated using BCA assay (Pierce, Rutherford, Ill.) and bovine serum albumin (BSA) as the standard.

TABLE 1
Primers used for PCR amplification of hOSM gene
and conducting point mutations thereafter.

| Gene/Mutation | | Sequence (5′ to 3′) | SEQ ID NO: |
|---|---|---|---|
| hOSM | FWD | CTGGTTCCGCGTGGATCCGCGGCTATAGGCAGC | 14 |
| | REV | CAGTCACGATGAATTCGACTATCTCCGGCTCCG | 15 |
| AGR to AGA | FWD | CACGAAGGCTGGCGCGGGGGCCTCTCAG | 16 |
| | REV | CTGAGAGGCCCCCGCGGCAGCCTTCGTG | 17 |
| FXXK to | FWD | CCCTGCCTCGGATGCTGCTCAGCGCGCGCTGGAGGGCTG | 18 |
| AXXA | REV | CAGCCCTCCAGCGCGCGCTGAGCAGCATCCGAGGCAGGG | 19 |
| OSM-M1 | FWD | GACTTAGAGCAGCGCCTCGGCGCGCCCCAGGATTTGGAGAGGT | 20 |
| (Round 1) | REV | ACCTCTCCAAATCCTGGGGCGCGCCGAGGCGCTGCTCTAAGTC | 21 |
| OSM-M1 | FWD | CTCGGCGCGCCCTCTGGGCTGAAC | 22 |
| (Round 2) | REV | GTTCAGCCCAGAGGGCGCGCCGAG | 23 |
| OSM-M2 | FWD | CAGCGCCTCGGCGGGGGCTCTGGGCTGAAC | 24 |
| (Round 1) | REV | GTTCAGCCCAGAGCCCCCGCCGAGGCGCTG | 25 |
| OSM-M2 | FWD | CCTCGGCGGGGGCAACATCGAGGACTT | 26 |
| (Round 2) | REV | AAGTCCTCGATGTTGCCCCCGCCGAGG | 27 |

Circular Dichroism: CD measurements were performed on a Jasco J-715 spectropolarimeter (Jasco, Easton, Mass.). Steady state spectra were recorded by scanning in the wavelength region between 200 and 250 nm with 0.1-cm path-length and a 1-nm bandwidth at 20° C. Spectra of blank buffer solutions acquired under identical conditions were used for background correction. Protein concentrations were maintained at 10 μM in Dulbecco's phosphate buffered saline (PBS) (9.33 mM potassium phosphate, 136 mM NaCl, 2.7 mM KCl, 0.6 mM MgCl₂, and 0.9 mM CaCl₂). Estimation of the α-helical, β-sheet and loop content in the proteins was carried out using SELCON3, CONTINNL and CDSSTR software programs (CD PRO®, Lamar, Colo.).

Surface Plasmon Resonance (SPR): Kinetic parameters of the interactions between receptor domains and the cytokines LIF, OSM-WT, OSM-M1 or OSM-M2 were analyzed by SPR using the SENSIQ® system (ICX Technologies, Oklahoma City, Okla.) as described by the manufacturer protocol. Briefly, a carboxyl sensor with two channels was installed in SENSIQ® and allowed to thermally equilibrate for about 15 minutes. The channels were initially cleaned with a 3 minute injection of 0.1 M HCl. An activation solution of 2 mM 1-Ethyl-3-[3-dimethylaminopropyl]carbodiimide hydrochloride (EDC) and 0.5 mM N-hydroxysulfo succinimide (NHS) was prepared in deionized water immediately before injection. Activation solution was injected over both channels for approximately 3 minutes followed by a 10 minute injection of 50 μg/mL cytokine (LIF, OSM-WT, OSM-M1 or OSM-M2) in 10 mM acetate buffer, pH 5.0, over channel 1. Channel 2 did not receive any cytokines and thus served as a reference for non-specific binding. Unreacted NHS esters were capped with a 3 minute injection of 1 M ethanolamine, pH 8.0, over both channels. Total immobilization of 500-700 RUs was achieved for each of these cytokines. A concentration series of soluble LIFR (# 249-LR-050/CF, R&D Systems, Minneapolis, Minn.), soluble OSMR (# 4389-OR-50, R&D Systems, Minneapolis, Minn.) or soluble gp130 (# 228-GP-050/CF, R&D Systems, Minneapolis, Minn.) in running buffer (10 mM HEPES pH 7.4, 150 mM NaCl, 3.4 mM EDTA, 0.005% Tween-20) were injected over both channels at a flow rate of 25 μl/min. Following a dissociation period of 3 minutes, the surfaces were regenerated by injecting 10 mM NaOH for 30 seconds. Rate constants for association (kₐ) and dissociation (k_{d}) rates were derived by global analysis of the response curves fit to a 1:1 kinetic model (Equations 1 and 2) using QDat software (BioLogic Software, Ltd. Knoxville Tenn. and Nomadics, Inc. Stillwater, Okla.) using 1:1 stoichiometry.

RU=RUₘₐₓe^{-k}^{_{d}}^{t} (Eqn 1)

RU=RUₘₐₓ(1−e^{-( k}^{ₐ}^{[C}^{ₒ}^{]+k}^{_{d}}^{)t}) (Eqn 2)

Where, RU—real time response units as measured by the SENSIQ® instrument; RUₘₐₓ—the maximum response obtainable for a given concentration of the soluble receptor; t—time; and Cₒ—concentration of the soluble receptor analyte in solution.

Cell Culture and cytokine stimulation: Müller cells and A375 melanoma cells were grown in DMEM-F12 and RPMI 1640, respectively, and supplemented with fetal bovine serum (10%) (Invitrogen, Carlsbad, Calif.), penicillin (100 U/ml) and streptomycin (100 μg/ml) (Invitrogen, Carlsbad, Calif.). Cells were seeded in a 10 cm tissue culture dish at a density of 100,000 cells/plate and allowed to grow in a 37° C. humidified atmosphere with 5% CO₂. When the cells reached 80% confluency, the culture medium was changed to fresh serum free media (DMEM-F12 or RPMI 1640 supplemented with penicillin (100 U/ml) and streptomycin (50 μg/ml)). Serum starvation was carried out for 30 minutes before stimulation with desired doses of OSM-WT, OSM-M1 or OSM-M2 for a period of 20 minutes. Following stimulation, cells were harvested for measurements of STAT3 and ERK-1/2 activation by Western blots.

Western Blots: Harvested cells were homogenized in a lysis buffer (50 mM Tris-HCl (pH 7.5), 150 mM NaCl, 5 mM EDTA, 1% (v/v) NP-40, 5% (v/v) glycerol, and protease inhibitor cocktail (Calbiochem, San Diego, Calif.). Protein content was measured using BCA protein assay (Pierce, Rutherford, Ill.). Total protein from each sample (15 μg) was electrophoresed on 4-20% gradient SDS-polyacrylamide gels (Invitrogen, Carlsbad, Calif.), and transferred to nitrocellulose membranes (Bio-Rad, Hercules, Calif.). The membranes were incubated in blocking buffer [5% BSA in TBST (20 mM Tris-HCl, pH 7.5, 100 mM NaCl, and 0.1% Tween-20)] for 1 hour at room temperature, and then incubated overnight at 4° C. with rabbit polyclonal anti-phospho STAT3 antibody (Cat # 9131, Cell Signaling Technology, Beverly, Mass.) or anti-phospho ERK-1/2 (Cat # 9101, Cell Signaling Technology, Beverly, Mass.) in blocking buffer, followed by a one hour incubation at room temperature with HRP-conjugated goat anti-rabbit secondary antibody (Cat # NA934V, GE Healthcare, UK Limited). Signals were visualized using SuperSignal West Dura extended duration substrate (Pierce, Rutherford, Ill.) and quantified by conventional digital image analysis using ImageStation 4000R (Software: Kodak MI; Eastman Kodak, Rochester, N.Y.). Blots were stripped and reprobed with anti-β-actin (Cat # ab6276-100, Abcam, Cambridge, Mass.) followed by appropriate secondary antibodies. Band intensities of pSTAT3 and pERK were normalized against the intensity of β-actin to account for loading variability.

Cell Proliferation Studies: To measure cell proliferation, ATP activity of the viable cells was quantified using CELLTITER-GLO® Luminescent cell viability assay (Promega, Madison, Wis.). A375 melanoma cells were seeded in a 96 well plate at a density of 4000 cells/well in a total volume of 200 μl of RPMI 1640 (Invitrogen, Carlsbad, Calif.) supplemented with fetal bovine serum (10%), penicillin (50 IU/ml) and streptomycin (50 mg/ml). Cells were then treated with different doses of OSM-WT, OSM-M1 or OSM-M2 for desired duration immediately after seeding. Control cells were treated with carrier solution, 1×PBS. Cell population in the wells was monitored using CELLTITER GLO® Luminescent cell viability assay (Promega, Madison, Wis.) according to the manufacturer's protocol.

Enzyme-linked immunosorbent assay (ELISA): ELISA was used to evaluate the equilibrium binding strength of the interaction between the cytokines and their receptors. This technique has traditionally been used as a sensitive method to quantify the binding affinities between two interacting proteins. However, unlike in SPR, ELISA involves immobilization of the proteins on a flat plastic surface driven by hydrophobic and ionic interactions. This might cause some distortion in the 3 dimensional structure of immobilized protein which leads to inaccuracies in the estimation of dissociation constants. It is thus important to bear in mind that the values estimated using this technique are to be used only for comparison between species but not as true equilibrium binding constants. The results obtained using this technique will thus be presented as apparent equilibrium dissociation constants (K_{D,App}). For direct interaction studies, cytokines (LIF, OSM-WT, OSM-M1 or OSM-M2) were immobilized on the 96 well ELISA plate by incubating the wells with 200 μl of 5 nM cytokine solution in PBS, pH 7.4 overnight at 4° C. The wells were then blocked with blocking buffer (4% BSA in PBS) for 1 hour at room temperature. After washing with 250 μl of washing buffer (0.05% Tween-20 in PBS) 3 times, the cytokines were treated with a series of concentrations of soluble human LIFR (Catalog #249-LR-050/CF, R&D Systems, Minneapolis, Minn.) or OSMR (Catalog #4389-OR-050, R&D Systems, Minneapolis, Minn.) in 150 μl of blocking buffer for 2 hours. The wells were then incubated with 150 μl of polyclonal anti-hLIFR (Catalog #AF249-NA, R&D Systems, Minneapolis, Minn.) or anti-hOSMR (Catalog #AF662, R&D Systems, Minneapolis, Minn.) in blocking buffer for 1 hour followed by incubation with 150 μl of HRP conjugated anti-mouse antibody (GE Healthcare, Uppsala, Sweden) in blocking buffer for 30 minutes. The wells were then washed 3 times with washing buffer and treated with 100 μl of chromogenic Slow-TMB® HRP substrate (Catalog #34024, Thermo Scientific Fisher, Rockford, Ill.) for 15 minutes. The reaction was then stopped by adding 100 μl of 2M H₂SO₄ and the absorbance of each well at 450 nm was read immediately using a UV detector (iMARK® Microplate Reader, Biorad, Hercules, Calif.). For interactions of higher order, soluble human gp130 (Catalog #671-GP-100, R&D Systems, Minneapolis, Minn.) was immobilized on 96 well ELISA microplates by incubating the wells with 200 μl of 1 nM gp130 solution (in PBS, pH 7.4) overnight at 4° C. The wells were then blocked with 150 μl of blocking buffer (4% BSA in PBS) for 1 hour at room temperature. After washing with 250 μl of washing buffer 3 times, gp130 was treated with saturating amounts of the cytokines (500 nM hLIF, 200 nM OSM-WT, 200 nM OSM-M1 or 200 nM OSM-M2) in a volume of 150 μl of blocking buffer for a period of 2 hours. The cytokine solution was discarded and a series of concentrations of soluble human LIFR or OSMR in a final volume of 150 μl blocking buffer were then added to the wells and the incubation was continued for another 1 hour. After washing with 250 μl of washing buffer 3 times, the wells were then incubated with 150 μl of polyclonal anti-hLIFR or anti-hOSMR in blocking buffer for 1 hour. After 3 washes with 250 μl of washing buffer, the wells were then incubated with 150 μl of HRP conjugated anti-mouse antibody in blocking buffer for 30 minutes. The wells were then washed again 3 times with washing buffer and treated with 100 μl of chromogenic Slow-TMB® HRP substrate for 30 minutes. The reaction was then stopped by adding 100 μl of 2M H₂SO₄, and the absorbance of each well at 450 nm was read using a UV detector. Equilibrium dissociation constants (K_{D}) were estimated by non-linear curve fitting to the optical density (OD) values plotted against the concentrations of soluble receptor using GraphPad Prism software (GraphPad Software, La Jolla, Calif.).

Statistical Analysis: All statistical analyses were done using SigmaStat 3.10 (Systat Software, Inc. Richmond, Calif.). Results are expressed as mean±standard deviation (SD). Differences between two groups were assessed using student t-test. A ‘p-value’ less than 0.05 were considered significant.

Results of Example 1

Molecular modeling of LIF and OSM; Identification of the BC loop. To determine the structural differences that might account for receptor specificity, the crystal structure of hOSM (PDB ID: 1EVS) was aligned onto hLIF (PDB ID: 1EMR) based on the trace of α-carbons using Delano Scientific's PyMol molecular viewer (http://pymol.org) (FIG. 1). The alignment of the backbone structures fit well with a relatively low RMSD value of 4.342. The active sites II and III on both molecules exhibited good conservation in structural orientation. Previous reports showed that the FXXK motif (SEQ ID NO:12) is essential for OSM's interaction with both LIFR and OSMR (Deller et al., 2000; Liu et al., 2009). In spite of having a similar FXXK motif, other LIFR interacting cytokines LIF, CNTF, CT-1 and CLC however cannot activate OSMR (Tanaka et al., 2003). This suggested that the difference in receptor specificity between hOSM and other LIFR activating cytokines is the result of structural differences between these ligands in the vicinity of the core FXXK motif (Deller et al., 2000). One of the obvious structural differences in the alignments is the presence of an additional helical loop between its B and C helices in OSM that is not present in LIF (FIG. 1). This BC loop is positioned in close proximity to the FXXK motif in active site III. Based on the crystal structures solved for LIF in complex with LIFR (PDB ID: 2Q7N) or gp130 (PDB ID: 1PVH), a model for the trimeric complex of LIF:LIFR:gp130 using PyMol was generated (FIG. 1D). When OSM was superimposed over LIF in this trimeric model, the BC loop on OSM again stands out as a unique motif at the receptor binding interface of OSM. This suggested that the BC loop is possibly playing an essential role in recognizing OSMR. To test this hypothesis, substitution mutations were generated in OSM that either remove or shorten the length of this BC loop.

Wild type OSM contains 12 amino acids in the BC loop region. Using site directed mutagenesis, the amino acids in this region were deleted or modified to generate OSM molecules that contained 7, 4 or 0 amino acids. Shown in FIG. 5 are the sequences of mutant OSM molecules with truncated BC loops (OSM-M1, OSM-M2 and OSM-M3) in comparison to the wild-type OSM (OSM-WT). Glycines (G) were incorporated into OSM-M1 and OSM-M2 to induce flexibility into the loop region, thus minimizing the impact of this BC loop modification on the overall structure of OSM. OSM-WT, OSM-M1 and OSM-M2 were expressed at high levels in bacteria, but OSM-M3 was not expressed in bacteria; this suggests that complete removal of the BC loop from OSM leads to instability in the overall structure of the protein (FIG. 4).

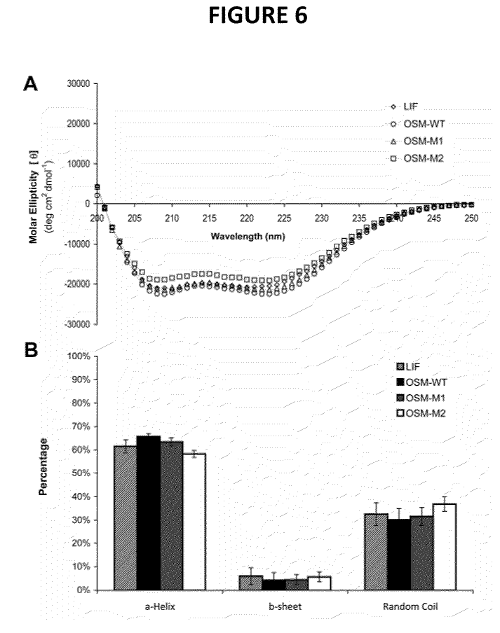

Structural characterization of OSM-Ml and OSM-M2. Minor alterations in the size and composition of the BC loop could potentially induce a global change in the overall structure of OSM. To determine whether the modified proteins (OSM-M1 and OSM-M2) still retained native alpha helical content, the recombinant proteins were analyzed using circular dichroism (CD). FIG. 6 shows the molar ellipticity [θ] plotted against the wavelength for LIF, OSM-WT, OSM-M1 and OSM-M2. All molecules displayed similar absorption behavior. Analysis using the software programs SELCON3, CONTINLL and CDSSTR revealed that both LIF and OSM have approximately 60% alpha helical content, with the remaining primarily being loop regions. This is in good agreement with the crystal structures available for LIF and OSM. Analysis also showed that both OSM-M1 and OSM-M2 have similar 60% alpha helical content with the remaining being loop regions. These results demonstrate that shortening the length of the BC loop in OSM from 12 amino acids to 7 or 4 amino acids did not induce a significant global change in the overall secondary structure of OSM.

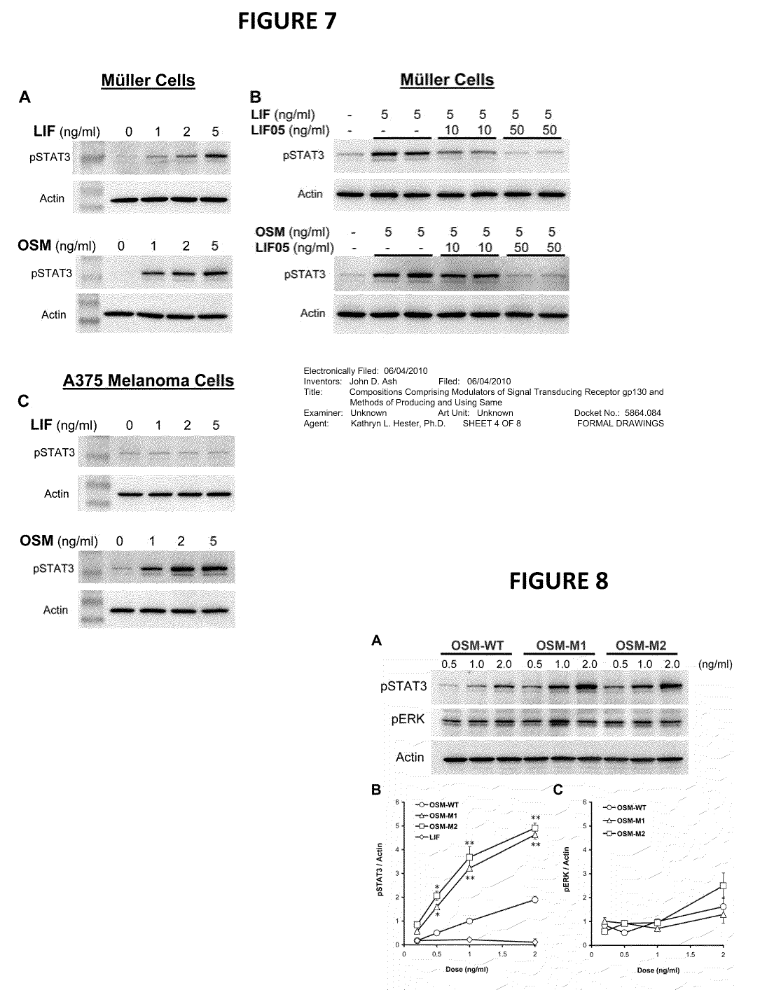

Functional Evaluation of wild-type and mutant OSMs in activating OSMR:gp130 complexes. To determine whether the BC loop on hOSM is required for OSMR binding, A375 melanoma cells were stimulated with the wild type and mutant forms of OSM. A375 melanoma cells express OSMR and gp130 on their cell surface, but do not express LIFR on their cell surface (Auguste et al., 1997). To confirm the absence of LIFR, A375 melanoma cells were treated with increasing doses of LIF and OSM. In agreement with previous observations, these cells did not respond to LIF, but responded to OSM in a linear, dose dependent manner as demonstrated by activation of STAT3 (FIGS. 7 and 8). When treated with OSM-M1 and OSM-M2, which have truncated BC loops, the A375 cells unexpectedly exhibited a 3-4 fold increase in their STAT3 activation relative to wild-type OSM (OSM-WT). The mutant molecules, however, did not show any change in Erk 1/2 activation compared to OSM-WT.

Functional Evaluation of wild-type and mutant OSMs in activating LIFR:gp130 complexes. To determine whether shortening the BC loop in OSM affected the ability of OSM to activate LIFR:gp130 receptors, the recombinant proteins were used to stimulate a human retinal Müller cell line. Müller cells respond to both LIF and OSM stimulation in a dose dependent manner by activating STAT3 (FIG. 7). To determine the receptor expression, the cells were pre-treated with recombinant LIF05 (a mutant LIF molecule that specifically antagonizes the activation of LIFR but not OSMR or gp130 (Hudson et al., 1996; Vernallis et al., 1997)), before stimulating with LIF or OSM. At doses of 50 ng/ml, LIF05 was able to completely antagonize the STAT3 activation induced by both LIF and OSM, demonstrating that the STAT3 activation in Müller cells is dependent upon utilization of LIFR:gp130 and not OSMR:gp130.

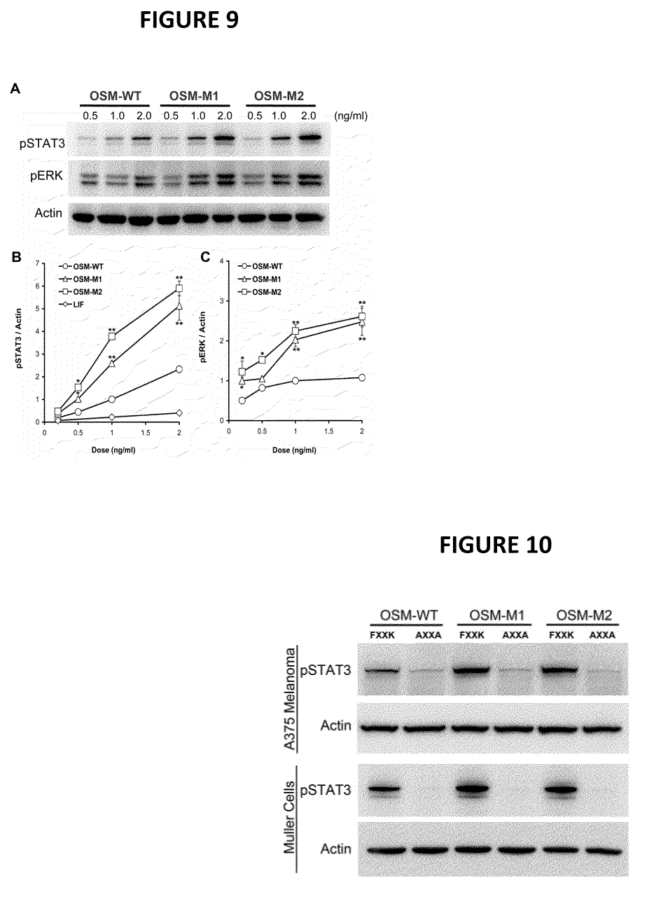

Treatment of Müller cells with wild-type and mutant OSM molecules again show that OSM-M1 and OSM-M2 induced a 2 to 3 fold greater activation of STAT3 compared to OSM-WT at similar doses (FIG. 9). Also, OSM-M1 and OSM-M2 exhibited a similar 2-3 fold higher activation of Erk1/2 compared to wild type OSM.

Removal of the BC loop does not alter the requirement of the core FXXK motif (SEQ ID NO:12) in active site III. Given its proximity to the active site III, it is possible that removal of the BC loop created an alternative site III that could facilitate a stronger binding to LIFR and OSMR. In order to determine whether the mutant OSMs, OSM-M1 and OSM-M2 still utilize the FXXK motif to interact with LIFR or OSMR, both F160 and K163 were mutated to alanines (A), and their activity on Müller cells and A375 cells was evaluated. Müller cells and A375 melanoma cells were serum starved for 30 minutes before stimulation with 1 ng/ml concentration of either the wild type (FXXK; SEQ ID NO:12) or the alanine mutant versions (AXXA; SEQ ID NO:13) of OSM-WT, OSM-M1 and OSM-M2 (FIG. 10). As expected, mutating FXXK to AXXA in OSM-WT completely abolishes its ability to activate STAT3 in both A375 melanoma and Müller cells. Similar to OSM-WT, both OSM-M1 and OSM-M2 showed complete loss of activity upon alanine substitution at the active site III.

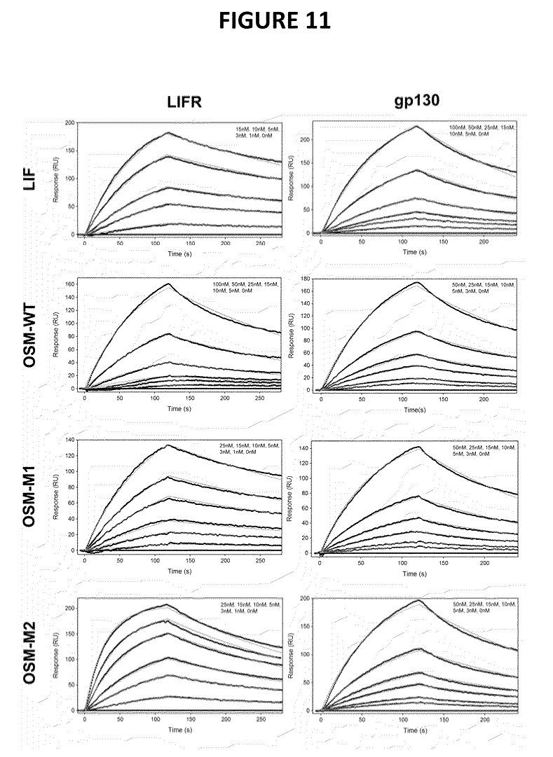

Reducing the size of the BC loop improves OSM's affinity towards LIFR and OSMR. Compared to OSM-WT, the higher potency of OSM-M1 and -M2 in terms of activating receptor signaling suggested that the BC loop on OSM is preventing OSM's ability to form a stable complex with the receptors. To directly measure the binding kinetics of these ligand-receptor interactions, surface plasmon resonance (SPR) was used. The cytokines (LIF, OSM-WT, OSM-M1 and OSM-M2) were immobilized on the sensor chip surface, while recombinant soluble receptors were used as the analytes (FIG. 11). The analysis revealed that LIF had a 23-fold higher affinity towards LIFR (KD=3.10 nM) than gp130 (KD=72.38 nM), while OSM had a 2-fold higher affinity towards gp130 (KD=22.69 nM) than LIFR (KD=43.79 nM) (Table 2). When the size of the BC loop was reduced from 12 aa to 7 aa (OSM-M1), the affinity of OSM toward LIFR improved dramatically (KD: 7.62 nM). When the size of the BC loop was further reduced to 4 aa (OSM-M2), the affinity improved even more (KD: 2.74 nM). However, changing the size of the BC loop did not affect OSM's affinity towards gp130 significantly (KD: OSM-WT=22.69 nM, OSM-M1=26.26 nM and OSM-M2=21.49 nM) (Table 2). OSM-M1 and -M2 proteins with shorter BC loops clearly displayed a higher affinity for LIFR while still retaining their relatively high affinity towards gp130. Together, these results demonstrate that the BC loop on OSM is presenting a steric hindrance for OSM's direct interaction with LIFR.

TABLE 2
Comparison of association (kₐ), dissociation (k_{d}) and equilibrium
dissociation (K_{D}) constants for LIFR and gp130 binding to LIF,
OSM-WT, OSM-M1 and OSM-M2

| | LIFR | | | gp130 | | |
|---|---|---|---|---|---|---|
| | kₐ | | | kₐ | | |
| | (×10⁵ | k_{d} | K_{D} | (×10⁵ | k_{d} | K_{D} |
| | M⁻¹s⁻¹) | (×10⁻³ s⁻¹) | (nM) | M⁻¹s⁻¹) | (×10⁻³ s⁻¹) | (nM) |
| LIF | 7.40 | 2.30 | 3.10 | 0.74 | 5.33 | 72.38 |
| OSM-WT | 0.91 | 4.00 | 43.79 | 2.07 | 4.70 | 22.69 |
| OSM-M1 | 3.31 | 2.52 | 7.62 | 2.03 | 5.34 | 26.26 |
| OSM-M2 | 13.0 | 3.56 | 2.74 | 2.27 | 4.87 | 21.49 |

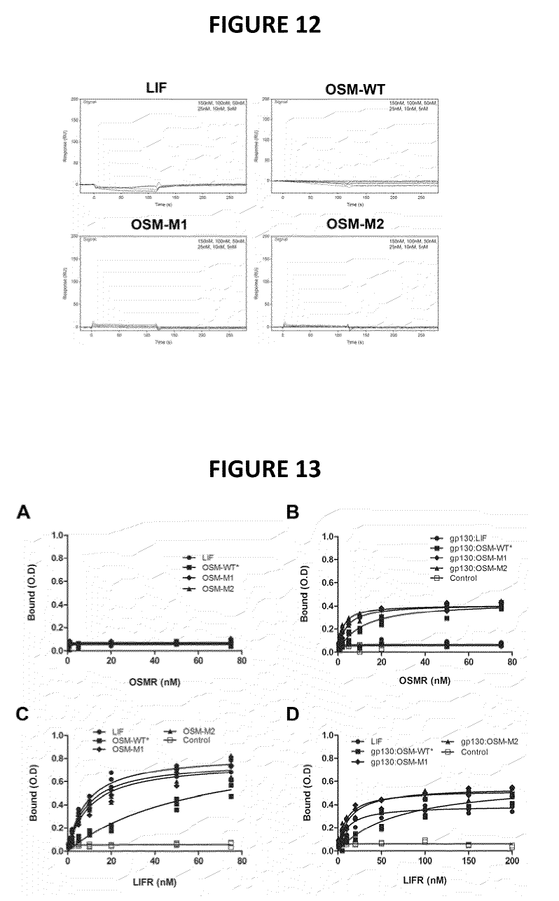

Similar binding studies with OSMR showed that neither the wild-type nor the mutant OSM's (OSM-M1 and OSM-M2) exhibited a direct interaction with OSMR (FIG. 12). This is in agreement with previous results, which reported a lack of direct interaction between OSM and OSMR in the absence of gp130 (Deller et al., 2000). In order to evaluate the binding kinetics of OSMR towards gp130 bound wild-type or mutant OSMs, soluble gp130 was immobilized on the sensor chip surface and treated with human OSM followed by OSMR. However, accurate association and dissociation constants could not be determined for these interactions, since there was a progressive loss in the binding capacity of gp130 with each round of binding and regeneration. To overcome this issue, ELISA was used, and similar binding assays were performed, where gp130 immobilized on the ELISA plate was sequentially treated with saturating amounts of wild-type or mutant OSMs followed by increasing concentrations of soluble OSMR. The results showed that after binding to gp130, both the wild type and the mutant OSMs started to exhibit a strong affinity towards OSMR (FIG. 13B; Table 3). However, when tested for direct interaction, none of these cytokines displayed detectable affinities towards OSMR (FIG. 13A; Table 3). This demonstrates that prior binding to gp130 is required for OSM to exhibit detectable affinities toward OSMR. Again, as observed towards LIFR, there was a significant improvement in OSM's affinity towards OSMR when the BC loop was truncated (K_{D,App}: OSM-WT—10.86±1.7 nM; OSM-M1—3.71±0.67 nM; OSM-M2—2.19±0.28 nM) (Table 3). This represents a 3-4 fold increase in the affinity towards OSMR upon BC loop truncation on OSM. These results again confirm that the BC loop on OSM presents a steric hindrance for both LIFR and OSMR interaction towards OSM, and its truncation resulted in significant improvement in both the affinity towards the receptors and also the biological activity of the molecule.

TABLE 3
Comparison of apparent equilibrium dissociation constant (K_{D,App})
values (nM) for direct interaction of LIFR and OSMR with LIF,
OSM-WT, OSM-M1 and OSM-M2 or the interaction of LIFR and
OSMR with gp130 bound LIF, OSM-WT, OSM-M1 and OSM-M2)

| | OSMR Binding | | LIFR Binding | |
|---|---|---|---|---|
| | Direct | With gp130 | Direct | With gp130 |
| LIF | ND | ND | 8.58 ± 0.99 | 10.33 ± 1.59 |
| OSM-WT* | ND | 10.86 ± 1.70 | 60.02 ± 17.54 | 70.29 ± 18.90 |
| OSM-M1 | ND | 3.71 ± 0.67 | 10.06 ± 1.34 | 12.56 ± 1.73 |
| OSM-M2 | ND | 2.19 ± 0.28 | 8.75 ± 1.34 | 9.13 ± 1.53 |

The gp130 induced co-operativity for OSM binding towards OSMR prompted the determination if a similar co-operativity was induced towards LIFR binding. However, these results revealed that prior gp130 binding to OSM or LIF did not affect their affinities towards LIFR significantly (Table 3). This demonstrates that, unlike in the case of OSMR, LIFR and gp130 bind to the cytokines in a non-cooperative manner. Again, as observed in SPR, the mutant OSMs with truncated BC loops exhibited a stronger affinity towards LIFR compared to the wild-type (FIGS. 13C and 13D, Table II). However, it is to be noticed that the equilibrium dissociation constants (K_{D,App}) obtained for LIFR binding using ELISA were significantly higher than the values obtained using SPR. This could be the result of possible structural distortions involved in ELISA immobilization and also the indirect method of binding analysis, which involves series of washing and incubation steps that shifts the equilibrium.

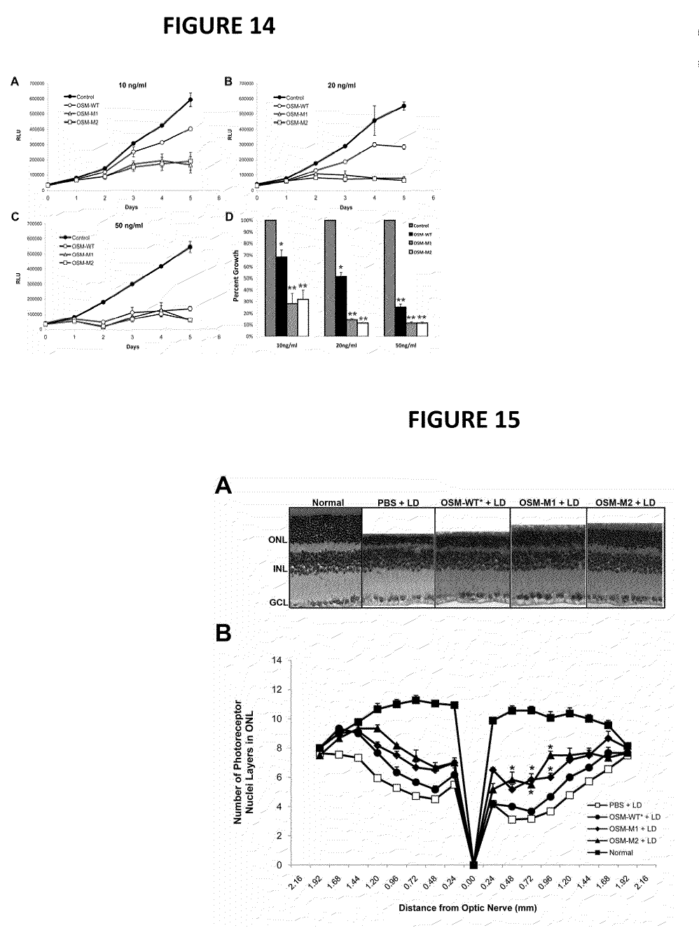

Inhibition of A375 melanoma cell proliferation. OSM was initially discovered by its ability to suppress proliferation of several melanoma cell lines, including A375 melanoma cells (Zarling et al., 1986). As expected, treating A375 cells with wild-type OSM inhibited their proliferation in a dose dependent manner (FIG. 14). At a concentration of 20 ng/ml, OSM-WT was able to suppress A375 melanoma cell proliferation by ˜50% while a concentration of 50 ng/ml was able to suppress the proliferation by ˜90%. In contrast, both OSM-M1 and OSM-M2 were both able to suppress the proliferation of A375 melanoma cells at significantly lower concentrations. While 10 ng/ml concentrations were enough for the mutant OSM molecules to inhibit the proliferation by ˜50%, 20 ng/ml concentrations suppressed their proliferation by ˜90%. These data demonstrate that reducing the size of the BC loop improves the ability of OSM to activate OSMR:gp130 and suppress the proliferation of A375 melanoma cells.

Discussion for Example 1

The members of the IL-6 family of cytokines are pleitropic cytokines that elicit a wide variety of responses in-vivo mediated by the activation of signal transducing receptors gp130, LIFR and OSMR. Among these cytokines, OSM is unique in terms of its ability to signal through two different receptor complexes, LIFR:gp130 (type I) and OSMR:gp130 (type II). Also, OSM is unique in the order in which it binds to its receptors, i.e., gp130 followed by LIFR or OSMR (Gearing et al., 1992; Tanaka et al., 2003; Sporeno et al., 1994; Liu et al., 1997). Based on the crystal structures and mutational analysis conducted, it has been proposed that the ability of OSM to interact with OSMR must result from the involvement of additional residues in the vicinity of its “FXXK” motif (SEQ ID NO:12), which is required for OSM's binding to LIFR and OSMR (Deller et al., 2000). In comparison to other members of the IL-6 family of cytokines, it has been identified herein that OSM has a unique α-helical loop between its B and C helices. This BC loop lies in close proximity to site III which contains the core “FXXK” motif. The size and location of this loop suggested that it is possibly playing an essential role in OSM's unique ability to bind OSMR. However, contrary to the expectations of the prior art, shortening this loop resulted in proteins that actually display higher activity, as indicated by improved activation of signal transduction and inhibition of A375 melanoma cell proliferation. Stimulation studies using Müller cells that express LIFR and gp130 showed that the truncation of the BC loop on OSM improves its ability to activate LIFR:gp130 complexes also. Kinetic and equilibrium binding analysis of ligand-receptor interaction revealed that the BC loop on OSM presents a steric hindrance for OSM's direct interaction with LIFR and OSMR, and shortening the loop results in dramatic improvement of its affinity towards either receptors. Together, these results demonstrate that the BC loop is clearly not essential for OSM's unique ability to bind OSMR, but is rather lowering the ability of OSM to form a stable complex with OSMR and gp130.

It has been reported that LIF has a strong preference for binding LIFR prior to binding gp130, while OSM has a preference for binding gp130 prior to binding LIFR (Gearing et al., 1992; Mosley et al., 1996; Hudson et al., 1996). Affinity measurements by SPR demonstrate that the mechanism behind the unique ability of OSM to first bind gp130 can be explained by its relative affinity to each receptor subunit. OSM has a two fold higher affinity towards gp130 than towards LIFR. LIF, which lacks the BC loop, has a 23 fold higher affinity for LIFR than for gp130. When the BC loop on OSM is truncated, OSM starts displaying a higher affinity towards LIFR than towards gp130. Clearly, the reduced affinity of OSM towards LIFR is caused by the BC loop and is likely playing a role in the difference in sequential binding between LIF and OSM.

The inability of OSM to bind soluble OSMR directly is consistent with previous observations (Deller et al., 2000). Like IL-6 and CNTF, which require binding to their alpha receptor before they can bind to their signal transducing receptors, these results demonstrate that OSM requires binding to gp130 before it can bind OSMR. ELISA analysis of OSMR binding towards the cytokines in the presence of gp130 showed that prior gp130 binding induces remarkable co-operativity towards OSMR binding in both the wild type and mutant OSMs (OSM-WT, OSM-M1 and OSM-M2). While the data clearly demonstrated that OSM utilizes the ‘FXXK’ motif for OSMR binding, binding to gp130 might expose otherwise hidden residues on OSM required for OSMR binding or alter the OSM structure to move hindering residues away from the binding interface, thereby leading to the strong binding. Solving the structure of OSM in complex with gp130 would prove valuable in identifying these changes.

Finally, a number of studies conducted over the last decade have revealed the diverse biological roles of OSM. One among them is the growth modulation of cells which include tumor cells, epithelial cells, fibroblasts and plasmacytoma cells (Zarling et al., 1986; Liu et al., 1997; Horn et al., 1990; Liu et al., 1998; Grant et al., 2001; Nishimoto et al., 1994). In agreement with earlier studies, the results of Example 1 demonstrated that OSM inhibits the growth of A375 melanoma cells in a dose dependent manner (FIG. 14). Mutant OSM proteins with shorter BC loops exhibited increased potency in suppressing their proliferation (FIG. 14). This improvement in OSM's function will prove valuable in treating diseases associated with melanoma. Previous research by the inventors has shown that STAT3 activation induced by members of the IL-6 family of cytokines, including OSM, is neuroprotective and prevents photoreceptor cell death under oxidative stress (Chollangi et al., 2009; Ueki et al., 2008). Example 2 below describes the evaluation of mutant OSM molecules as potent therapeutic agents in preventing photoreceptor degeneration induced by oxidative stress, e.g., retinitis pigmentosa. Also, OSM plays a key role in inflammatory response to injury and infection. OSM secreted from activated T cells and monocytes stimulates expression of: (1) acute phase proteins (APPs) in liver (Richards et al., 1992); (2) P-selectin and E-selectin on endothelial cells (Yao et al., 1996; Modur et al., 1997); and (3) TIMP-1 in fibroblasts (Richards et al., 1997; Richards et al., 1993), all of which promote wound repair. The mutant OSM proteins thus have therapeutic application in promoting wound healing also.

### Example 2

As described herein before, members of the IL-6 family of cytokines are strongly implicated in neuroprotection. Previous studies by LaVail et al., (1992) and Ueki et al., (2008) have shown that members of the IL-6 family of cytokines rescue photoreceptors from damaging effects of constant light and retinal degenerations induced by inherited genetic mutations. To test whether the mutant OSM's (OSM-M1 and OSM-M2), which display improved ability in binding and activating LIFR:gp130 and OSMR:gp130 complexes, exhibit an enhanced ability in protecting the retinal photoreceptors compared to wild-type OSM, 0.25 μg of OSM-WT, OSM-M1 or OSM-M2 in a total volume of 1 μl was injected in the left eye of Balb/cJ mice. The right eye was injected with PBS and served as a control. Two days following intravitreal injection, the mice were exposed to damaging light (4000 lux) for 4 hours. Following a 4 day recovery period after the light damage, eyes were enucleated and processed for histological analysis. Photoreceptor cell death in each group was assessed by morphometric analysis. Representative sections for each treatment are shown in FIG. 15A, while quantitative analysis of the number of photoreceptors in each group is shown in FIG. 15B. Exposure to 4000 lux for 4 hours caused significant damage to retinal photoreceptors injected with PBS. Compared to normal eyes, which contain ˜12 rows of photoreceptor nuclei in their outer nuclear layer (ONL), PBS injected eyes retained only 3-4 nuclei layers in the ONL. While OSM-WT did not show significant protection of photoreceptors, OSM-M1 and OSM-M2 were clearly more potent and lead to retainment of 6-7 photoreceptor nuclei layers after the light damage. Again, this demonstrates that the improved affinity of OSM towards OSMR and LIFR by truncating the BC loop has a functional consequence.

Thus, in accordance with the present invention, there have been provided compositions comprising functionally active modulators of gp130, as well as methods of producing and using same, that fully satisfy the objectives and advantages set forth hereinabove. Although the invention has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference.
Adamson, R. (2009) J. Wound. Care, 18:349-351
Adler, R. (1993) Curr. Opin. Neurobiol., 3:785-789
Ahima, R. S. (2006) Nat. Med., 12:511-512
Al-Khalili, L., et al. (2006) Mol. Endocrinol., 20:3364-3375
Anderson, K. D., et al. (1996) Proc. Natl. Acad. Sci. U.S.A., 93:7346-7351
Auguste, P., et al. (1997) J. Biol. Chem., 272:15760-15764
Barton, B. E. (1997) Clin. Immunol. Immunopathol., 85:16-20
Bell, M. C., et al. (1999) Arthritis Rheum., 42:2543-2551
Boniface, K., et al. (2007) J. Immunol., 178:4615-4622
Bravo, J. and Heath, J. K. (2000) EMBO J., 19:2399-2411
Brown, T. J., et al. (1991) J. Immunol., 147:2175-2180
Cao, W., et al. (1997) Exp. Eye Res., 65:241-248
Carey, Al. L., et al. (2006) Diabetes, 55:2688-2697
Carroll, G. J., et al. (1995) J. Interferon Cytokine Res., 15:567-573
Carroll, G., et al. (1998) Inflamm. Res., 47:1-7
Cawston, T. E. and Billington, C. (1996) J. Pathol., 180:115-117
Cawston, T., et al. (1999) Ann. N.Y. Acad. Sci., 878:120-129
Cawston, T., et al. (1994) Ann. N.Y. Acad. Sci., 732:75-83
Chen, R. W. and Postlethwait, R. W. (1964) Monogr Surg. Sci., 16:215-276
Chen, S. H., et al. (2006) Glia, 53:191-200
Choi, J. S., et al. (2003) Glia, 41:237-246
Chollangi, S., et al. (2009) Neurobiol. Dis., 34:535-544
Clark, I. M., et al. (1994) Biochem. Biophys. Res. Commun., 203:874-880
Cornish, J., et al. (1997) Bone, 21:243-247
Cornish, J., et al. (1993) Endocrinology, 132:1359-1366
Darnell, J. E., Jr. et al. (1994) Science, 264:1415-1421
Davis, S., et al. (1993) Science, 260:1805-1808
Davis, S., et al. (1991) Science, 253:59-63
De, B. F., et al. (1994) J. Clin. Invest, 93:2114-2119
De, B. F., et al. (1991) Arthritis Rheum., 34:1158-1163
De, B. F., et al. (1992) Clin. Exp. Rheumatol., 10:493-498
DeChiara, T. M., et al. (1995) Cell, 83:313-322
Deller, M. C., et al. (2000) Structure, 8:863-874
Di, M. A., et al. (1996) Proc. Natl. Acad. Sci. U.S.A., 93:9247-9252
Ellis, A. J., et al. (1994) Biochem. Biophys. Res. Commun., 201:94-101
Emerich, D. F., et al. (1997) Nature, 386:395-399
Gabay, C., et al. (1996) Clin. Exp. Immunol., 105:260-265
Gamido-Urbani, S., et al. (2008) Biochem. Soc. Trans., 36:203-211
Gearing, D. P., et al. (1992) Science, 255:1434-1437
Giovannini, M., et al. (1993) Cytogenet. Cell Genet., 64:240-244
Giovannini, M., et al. (1993) Cytogenet. Cell Genet., 62:32-34
Glaros, T., et al. (2009) Front Biosci., 14:3988-3993
Gloaguen, I., et al. (1997) Proc. Natl. Acad. Sci. U.S.A., 94:6456-6461
Grant, S. L., et al. (2001) Growth Factors, 19:153-162
Grenier, A., et al. (1999) Blood, 93:1413-1421
Grimm, C., et al. (2005) Semin. Cell Dev. Biol., 16:531-538
Grimm, C., et al. (2002) Nat. Med., 8:718-724
Guerne, P. A., et al. (1989) J. Clin. Invest., 83:585-592
Halfter, H., et al. (1998) Growth Factors, 15:135-147
Hamilton, J. A., et al. (1993) J. Immunol., 150:1496-1502
Heinrich, P. C., et al. (1998) Biochem. J., 334:(Pt 2)297-314
Heinrich, P. C., et al. (1995) Ann. N.Y. Acad. Sci., 762:222-236
Hilton, D. J., et al. (1994) EMBO J., 13:4765-4775
Hoenig, M. R., et al. (2008) Curr. Drug Targets., 9:422-435
Horn, D., et al. (1990) Growth Factors, 2:157-165
Houssiau, F. A., et al. (1988) Arthritis Rheum. 31:784-788
Hudson, K. R., et al. (1996) J. Biol. Chem., 271:11971-11978
Hui, W., et al. (1997) Ann. Rheum. Dis., 56:184-187
Ichihara, M., et al. (1997) Blood, 90:165-173
Jeffery, E., et al. (1993) Cytokine, 5:107-111
Joly, S., et al. (2008) J. Neurosci., 28:13765-13774
Kaibara, A., et al. (1998) Cytokine, 10:452-456
Koj, A., et al. (1991) Biomed. Biochim. Acta, 50:421-425
Koj, A., et al. (1992) Folia Histochem. Cytobiol., 30:143-145
Kordula, T., et al. (1991) Lymphokine Cytokine Res., 10:23-26
Korpos, E., et al. (2009) Curr. Pharm. Des., 15:1349-1357
Korpos, E., et al. (2009) Cell Tissue Res.
Korzus, E., et al. (1997) J. Biol. Chem., 272:1188-1196
Kotake, S., et al. (1996) J. Bone Miner. Res., 11:88-95
Lambert, P. D., et al. (2001) Proc. Natl. Acad. Sci. U.S.A., 98:4652-4657
LaVail, M. M., et al. (1991) Ann. N.Y. Acad. Sci., 638:341-347
LaVail, M. M., et al. (1992) Proc. Natl. Acad. Sci. U.S.A., 89:11249-11253
LaVail, M. M., et al. (1998) Invest Opthalmol. Vis. Sci., 39:592-602
Li, F., et al. (2001) Exp. Eye Res., 73:569-577
Li, F., et al. (2003) Invest Opthalmol. Vis. Sci., 44:4968-4975
Li, M., et al. (1995) Nature, 378:724-727
Linker, R. A., et al. (2002) Nat. Med., 8:620-624
Liu, C., et al. (1998) J. Neurosci., 18:1337-1344
Liu, H., et al. (2009) Int. J. Mol. Med., 23:161-172
Liu, J., et al. (1998) Cytokine, 10:295-302
Liu, J., et al. (1992) J. Biol. Chem., 267:16763-16766
Liu, J., et al. (1997) Cell Growth Differ., 8:667-676
Lotz, M., et al. (1992) J. Clin. Invest., 90:888-896
Lowe, C., et al. (1991) J. Bone Miner. Res., 6:1277-1283
Lowe, C., et al. (1991) Calcif. Tissue Int., 49:394-397
Lutticken, C., et al. (1994) Science, 263:89-92
Marcos-Gomez et al. (2008) An Sist Sanit Navar., 31(2):113-23.
Modur, V., et al. (1997) J. Clin. Invest., 100:158-168
Mosley, B., et al. (1996) J. Biol. Chem., 271:32635-32643
Murakami, M., et al. (1993) Science, 260:1808-1810
Murphy, M., et al. (1994) Development, 120:3519-3528
Murphy, M., et al. (1991) Proc. Natl. Acad. Sci. U.S.A., 88:3498-3501
Nesbitt, J. E., et al. (1993) Biochem. Biophys. Res. Commun., 190:544-550
Nishimoto, N., et al. (1994) J. Exp. Med., 179:1343-1347
Ochoa, O., et al. (2007) Vascular., 15:350-355
Okamoto, H., et al. (1997) Arthritis Rheum., 40:1096-1105
Rattner, A. and Nathans, J. (2005) J. Neurosci., 25:4540-4549
Rattner, A., et al. (2008) J. Neurosci., 28:9880-9889
Reid, L. R., et al. (1990) Endocrinology, 126:1416-1420
Richards, C. D., et al. (1992) J. Immunol., 148:1731-1736
Richards, C. D., et al. (1997) J. Immunol., 159:2431-2437
Richards, C. D., et al. (1993) J. Immunol., 150:5596-5603
Robak, T., et al. (1998) Mediators. Inflamm., 7: 347-353
Robledo, O., et al. (1997) J. Biol. Chem., 272:4855-4863
Rokita, H., et al. (1992) Scand. J. Immunol., 35:681-685
Rose, T. M., et al. (1993) Genomics, 17:136-140
Saharinen, P., et al. (2008) Nat. Cell Biol., 10:527-537
Schooltink, H., et al. (1991) Biochem. J., 277(Pt 3):659-664
Sendtner, M., et al. (1991) J. Cell Sci. Suppl., 15:103-109
Sendtner, M., et al. (1990) Nature, 345:440-441
Sendtner, M., et al. (1992) Nature, 358:502-504
Simpson, R. J., et al. (1997) Protein Sci., 6:929-955
Sporeno, E., et al. (1994) J. Biol. Chem., 269:10991-10995
Stahl, N., et al. (1994) Science, 263:92-95
Stahl, N., et al. (1995) Science, 267:1349-1353
Stewart, C. L., et al. (1992) Nature, 359:76-79
Taga, T., et al. (1992) Chem. Immunol., 51:181-204
Takagi, N., et al. (1998) Arthritis Rheum., 41:2117-2121
Tanaka, M. and Miyajima, A. (2003) Rev. Physiol Biochem. Pharmacol., 149:39-52
Teller, P. and White, T. K. (2009) Surg. Clin. North Am., 89:599-610
Ueki, Y., et al. (2008) J. Neurochem., 105:784-796
Vernallis, A. B., et al. (1997) J. Biol. Chem., 272:26947-26952
Vestweber, D., et al. (2009) Trends Cell Biol., 19:8-15
Ware, C. B., et al. (1995) Development, 121:1283-1299
Waring, P. M., et al. (1993) Arthritis Rheum., 36:911-915
Watt, M J., et al. (2006) Nat. Med., 12:541-548
Watt, M. J., et al. (2006) Endocrinology, 147:2077-2085
Weiss, T. W., et al. (2006) FASEB J., 20:2369-2371
Woessner, J. F., Jr. (1991) FASEB J., 5:2145-2154
Yager, D. R., et al. (2007) Int. J. Low Extrem. Wounds., 6:262-272
Yamasaki, K., et al. (1988) Science, 241:825-828
Yao, L., et al. (1996) J. Exp. Med., 184:81-92
Zarling, J. M., et al. (1986) Proc. Natl. Acad. Sci. U.S.A., 83:9739-9743

What is claimed is:

1. A composition comprising a functionally active modulator for gp130, wherein the functionally active modulator comprises:
a variant of Oncostatin M (OSM), whereby activation of gp130 by the variant is increased when compared to activation of gp130 by wild type OSM, wherein the OSM variant has at least one amino acid sequence truncation, deletion or substitution in a BC loop thereof when compared to wild type OSM, and wherein said modification to the BC loop decreases steric hindrance and increases affinity of the variant for at least one receptor selected from the group consisting of leukemia inhibitory factor (LIFR) and Oncostatin M receptor (OSMR) when compared to wild type OSM.

2. The composition of claim 1, whereby the activation of gp130 results in increased activation of STAT3 and MAPK pathways when compared to activation of gp130 by wild type OSM.

3. The composition of claim 1, wherein at least one of:
(a) the variant comprises at least one mutation in at least one protease cleavage site in the amino acid sequence of wild type OSM, thereby rendering the modulator resistant to protease cleavage;
(b) the wild type OSM comprises the amino acid sequence of SEQ ID NO:1, and the variant has at least one amino acid sequence truncation, deletion or substitution in SEQ ID NO:9 of SEQ ID NO:1;
(c) the variant comprises an amino acid sequence selected from the group consisting of SEQ ID NOS:6-8;
(d) an amino acid sequence of the variant comprises at least one amino acid sequence substitution, addition and/or deletion when compared to SEQ ID NO:1 and comprises the motifs of SEQ ID NOS:2-5 and 12;
(e) an amino acid sequence of the OSM variant is at least 90% identical to SEQ ID NO:1.

4. The composition of claim 1, further defined as a pharmaceutical composition.

5. The composition of claim 4, further comprising a pharmaceutically acceptable carrier.

6. An isolated and purified nucleic acid segment encoding a variant of Oncostatin M, whereby the variant exhibits increased activation of gp130 when compared to native OSM, and wherein the OSM variant has at least one amino acid sequence truncation, deletion or substitution in a BC loop thereof when compared to wild type OSM, the nucleic acid segment comprising at least one of:
(a) a nucleic acid segment encoding an amino acid sequence selected from the group consisting of SEQ ID NOS:6-8;
(b) a nucleic acid segment encoding an amino acid sequence that comprises at least one amino acid sequence substitution, addition and/or deletion when compared to SEQ ID NO:1 and comprises the motifs of SEQ ID NOS:2-5 and 12;
(c) a nucleic acid segment encoding an amino acid sequence that comprises at least one amino acid sequence truncation, deletion or substitution in SEQ ID NO:1, and wherein said at least one amino acid sequence truncation, deletion or substitution occurs in SEQ ID NO:9 of SEQ ID NO:1;
(d) a nucleic acid segment encoding an amino acid sequence that is at least 90% identical to SEQ ID NO:1; and
(e) a nucleic acid segment encoding an amino acid sequence that comprises at least one amino acid substitution, addition and/or deletion when compared to SEQ ID NO:1, wherein said at least one substitution, addition and/or deletion occurs in a protease cleavage site of SEQ ID NO:1, whereby the resultant polypeptide is resistant to protease cleavage.

7. A recombinant vector comprising the nucleic acid segment of claim 6.

8. A recombinant host cell comprising the recombinant vector of claim 7.

9. A method of producing a functionally active modulator of gp130, comprising the steps of:
providing a host cell encoding a variant of Oncostatin M, whereby the variant exhibits increased activation of gp130 when compared to native OSM, and wherein the OSM variant has at least one amino acid sequence truncation, deletion or substitution in a BC loop thereof when compared to wild type OSM; and
culturing the host cell under conditions that allow for production of the OSM variant.

10. The method of claim 9, wherein at least one of:
(a) wild type OSM comprises the amino acid sequence of SEQ ID NO:1, and the at least one amino acid sequence truncation, deletion or substitution in the BC loop of the OSM variant occurs in SEQ ID NO:9 of SEQ ID NO:1;
(b) the OSM variant comprises an amino acid sequence selected from the group consisting of SEQ ID NOS:6-8;
(c) an amino acid sequence of the OSM variant comprises at least one amino acid sequence substitution, addition and/or deletion when compared to SEQ ID NO:1 and comprises the motifs of SEQ ID NOS:2-5 and 12;
(d) an amino acid sequence of the OSM variant is at least 90% identical to SEQ ID NO:1; and
(e) the OSM variant comprises at least one mutation in at least one protease cleavage site in the amino acid sequence of the native ligand, thereby rendering the modulator resistant to protease cleavage.

11. A method of activating at least one gp130 signaling cascade, the method comprising the steps of:
providing at least one cell having gp130 expressed on a surface thereof;
administering an effective amount of a functionally active modulator of gp130 to the at least one cell, the functionally active modulator of gp130 comprising a variant of Oncostatin M (OSM), wherein the variant has at least one amino acid sequence truncation, deletion or substitution in a BC loop thereof when compared to wild type OSM; and
whereby the functionally active modulator binds to gp130 and causes gp130 to homo-dimerize or hetero-dimerize with another receptor on the surface of the at least one cell, whereby the binding and dimerization activates at least one gp130 signaling cascade.

12. The method of claim 11, wherein at least one of:
(a) wild type OSM comprises the amino acid sequence of SEQ ID NO:1, and the at least one amino acid sequence truncation, deletion or substitution in the BC loop of the OSM variant occurs in SEQ ID NO:9 of SEQ ID NO:1;
(b) the OSM variant comprises an amino acid sequence selected from the group consisting of SEQ ID NOS:6-8;
(c) an amino acid sequence of the OSM variant comprises at least one amino acid sequence substitution, addition and/or deletion when compared to SEQ ID NO:1 and comprises the motifs of SEQ ID NOS:2-5 and 12;
(d) an amino acid sequence of the OSM variant is at least 90% identical to SEQ ID NO:1; and
(e) the OSM variant comprises at least one mutation in at least one protease cleavage site in the amino acid sequence of the native ligand, thereby rendering the modulator resistant to protease cleavage.

13. A method for providing neuroprotection to a patient in need thereof, the method comprising the step of:
administering to the patient a therapeutically effective amount of a pharmaceutical composition comprising a functionally active modulator for gp130, the functionally active modulator for gp130 comprising a variant of Oncostatin M (OSM), wherein the variant has at least one amino acid sequence truncation, deletion or substitution in a BC loop thereof when compared to wild type OSM.

14. The method of claim 13, further defined as a method for providing retinal neuroprotection to a patient, and wherein the pharmaceutical composition is administered to at least one eye of the patient.

15. The method of claim 14, wherein the neuroprotective effect diminishes, or protects the subject from, at least one of neuronal damage caused by exposure to oxidative stress, neuronal damage caused by light stress, and retinal degeneration induced by inherited genetic mutation.

16. A method of treating a disorder in a mammal in need of such treatment, comprising the step of:
administering a therapeutically effective amount of a pharmaceutical composition comprising a functionally active modulator for gp130, the functionally active modulator for gp130 comprising a variant of Oncostatin M (OSM), wherein the variant has at least one amino acid sequence truncation, deletion or substitution in a BC loop thereof when compared to wild type OSM.

17. The method of claim 16, wherein the disorder is selected from the group consisting of age related degenerations, progressive degenerations, acute pancreatitis, Alzheimer's disease, generically inherited mutations, obesity, diabetes, insulin resistance, glucose intolerance, dyslipidemia, hypertension, hypercholesterolemia, cancer, melanoma, inflammation and inflammatory disorders, inflammatory arthropathy, gout, rheumatoid arthritis, osteoarthritis, inflammatory vascular diseases, injury, infection, infertility, haematopoietic disorders, angiogenetic disorders, and combinations thereof.

18. The method of claim 16, wherein at least one of:
(a) wild type OSM comprises the amino acid sequence of SEQ ID NO:1, and the at least one amino acid sequence truncation, deletion or substitution in the BC loop of the OSM variant occurs in SEQ ID NO:9 of SEQ ID NO:1;
(b) the OSM variant comprises an amino acid sequence selected from the group consisting of SEQ ID NOS:6-8;
(c) an amino acid sequence of the OSM variant comprises at least one amino acid sequence substitution, addition and/or deletion when compared to SEQ ID NO:1 and comprises the motifs of SEQ ID NOS:2-5 and 12;
(d) an amino acid sequence of the OSM variant is at least 90% identical to SEQ ID NO:1; and
(e) the OSM variant comprises at least one mutation in at least one protease cleavage site in the amino acid sequence of the native ligand, thereby rendering the modulator resistant to protease cleavage.
